(19) United States
(12) Patent Application Publication
(10) Pub. No.: US 2010/0137440 A1
(43) Pub. Date: Jun. 3, 2010

(54) LYSINE RIBOSWITCHES, STRUCTURE-BASED COMPOUND DESIGN WITH LYSINE RIBOSWITCHES, AND METHODS AND COMPOSITIONS FOR USE OF AND WITH LYSINE RIBOSWITCHES

(75) Inventors: Ronald R. Breaker, Guilford, CT (US); Jinsoo Lim, Hamden, CT (US); Kenneth F. Blount, Guilford, CT (US); Joy Wang, New Haven, CT (US); Narasimhan Sudarsan, New Haven, CT (US)

Correspondence Address: PATENT CORRESPONDENCE;ARNALL GOLDEN GREGORY LLP, 171 17TH STREET NW, SUITE 2100, ATLANTA, GA, 30363, US

(73) Assignee: Yale University, New Haven, CT (US)

(21) Appl. No.: 12/440,808

(22) PCT Filed: Sep. 11, 2007
(86) PCT No.: PCT/US07/78195

§ 371 Date: Jan. 22, 2010

Related U.S. Application Data
(60) Provisional application No. 60/843,728, filed on Sep. 11, 2006.

(51) Int. Cl. A61K 31/195; C12N 1/20; C12Q 1/68

(52) U.S. Cl. 514/562; 435/252.1; 514/564; 514/565; 435/6

(57) ABSTRACT

The lysine riboswitch is a target for antibiotics and other small molecule therapies. Compounds can be used to stimulate, active, inhibit and/or inactivate the lysine riboswitch.

### CROSS-REFERENCE TO RELATED APPLICATIONS

This application claims benefit of U.S. Provisional Application No. 60/843,728, filed Sep. 11, 2006. U.S. Provisional Application No. 60/843,728, filed Sep. 11, 2006, is hereby incorporated herein by reference in its entirety.

### STATEMENT REGARDING FEDERALLY SPONSORED RESEARCH

This invention was made with government support under Grant No. W911NF-04-1-0416 awarded by DARPA; and Grant NIH R33-DK070270 awarded by the NIH. The government has certain rights in the invention.

### FIELD OF THE INVENTION

The disclosed invention is generally in the field of gene expression and specifically in the area of regulation of gene expression.

### BACKGROUND OF THE INVENTION

Precision genetic control is an essential feature of living systems, as cells must respond to a multitude of biochemical signals and environmental cues by varying genetic expression patterns. Most known mechanisms of genetic control involve the use of protein factors that sense chemical or physical stimuli and then modulate gene expression by selectively interacting with the relevant DNA or messenger RNA sequence. Proteins can adopt complex shapes and carry out a variety of functions that permit living systems to sense accurately their chemical and physical environments. Protein factors that respond to metabolites typically act by binding DNA to modulate transcription initiation (e.g. the lac repressor protein; Matthews, K. S., and Nichols, J. C., 1998, Prog. Nucleic Acids Res. Mol. Biol. 58, 127-164) or by binding RNA to control either transcription termination (e.g. the PyrR protein; Switzer, R. L., et al., 1999, Prog. Nucleic Acids Res. Mol. Biol. 62, 329-367) or translation (e.g. the TRAP protein; Babitzke, P., and Gollnick, P., 2001, J. Bacteriol. 183, 5795-5802). Protein factors respond to environmental stimuli by various mechanisms such as allosteric modulation or post-translational modification, and are adept at exploiting these mechanisms to serve as highly responsive genetic switches (e.g. see Ptashne, M., and Gann, A. (2002). Genes and Signals. Cold Spring Harbor Laboratory Press, Cold Spring Harbor, N.Y.).

In addition to the widespread participation of protein factors in genetic control, it is also known that RNA can take an active role in genetic regulation. Recent studies have begun to reveal the substantial role that small non-coding RNAs play in selectively targeting mRNAs for destruction, which results in down-regulation of gene expression (e.g. see Hannon, G. J. 2002, Nature 418, 244-251 and references therein). This process of RNA interference takes advantage of the ability of short RNAs to recognize the intended mRNA target selectively via Watson-Crick base complementation, after which the bound mRNAs are destroyed by the action of proteins. RNAs are ideal agents for molecular recognition in this system because it is far easier to generate new target-specific RNA factors through evolutionary processes than it would be to generate protein factors with novel but highly specific RNA binding sites.

Although proteins fulfill most requirements that biology has for enzyme, receptor and structural functions, RNA also can serve in these capacities. For example, RNA has sufficient structural plasticity to form numerous ribozyme domains (Cech & Golden, Building a catalytic active site using only RNA. In: The RNA World R. F. Gesteland, T. R. Cech, J. F. Atkins, eds., pp. 321-350 (1998); Breaker, In vitro selection of catalytic polynucleotides. Chem. Rev. 97, 371-390 (1997)) and receptor domains (Osborne & Ellington, Nucleic acid selection and the challenge of combinatorial chemistry. Chem. Rev. 97, 349-370 (1997); Hermann & Patel, Adaptive recognition by nucleic acid aptamers. Science 287, 820-825 (2000)) that exhibit considerable enzymatic power and precise molecular recognition. Furthermore, these activities can be combined to create allosteric ribozymes (Soukup & Breaker, Engineering precision RNA molecular switches. Proc. Natl. Acad. Sci. USA 96, 3584-3589 (1999); Seetharaman et al., Immobilized riboswitches for the analysis of complex chemical and biological mixtures. Nature Biotechnol. 19, 336-341 (2001)) that are selectively modulated by effector molecules.

Bacterial riboswitch RNAs are genetic control elements that are located primarily within the 5′-untranslated region (5″-UTR) of the main coding region of a particular mRNA. Structural probing studies (discussed further below) reveal that riboswitch elements are generally composed of two domains: a natural aptamer (T. Hermann, D. J. Patel, Science 2000, 287, 820; L. Gold, et al., Annual Review of Biochemistry 1995, 64, 763) that serves as the ligand-binding domain, and an ‘expression platform’ that interfaces with RNA elements that are involved in gene expression (e.g. Shine-Dalgarno (SD) elements; transcription terminator stems). What is needed in the art are methods and compositions that can be used to regulate lysine riboswitches.

### BRIEF SUMMARY OF THE INVENTION

It has been discovered that certain natural mRNAs serve as metabolite-sensitive genetic switches wherein the RNA directly binds a small organic molecule. This binding process changes the conformation of the mRNA, which causes a change in gene expression by a variety of different mechanisms. The natural switches are targets for antibiotics and other small molecule therapies.

Disclosed are compounds, and compositions containing such compounds, that can activate, deactivate or block the lysine riboswitch. Also disclosed are compositions and methods for activating, deactivating or blocking the lysine riboswitch. Riboswitches function to control gene expression through the binding or removal of a trigger molecule. Compounds can be used to activate, deactivate or block a riboswitch. The trigger molecule for a riboswitch (as well as other activating compounds) can be used to activate a riboswitch. Compounds other than the trigger molecule generally can be used to deactivate or block a riboswitch. Riboswitches can also be deactivated by, for example, removing trigger molecules from the presence of the riboswitch. A riboswitch can be blocked by, for example, binding of an analog of the trigger molecule that does not activate the riboswitch.

Also disclosed are compositions and methods for altering expression of an RNA molecule, or of a gene encoding an RNA molecule, where the RNA molecule includes a lysine riboswitch, by bringing a compound into contact with the RNA molecule. Riboswitches function to control gene expression through the binding or removal of a trigger molecule. Thus, subjecting an RNA molecule of interest that includes a lysine riboswitch to conditions that activate, deactivate or block the riboswitch can be used to alter expression of the RNA. Expression can be altered as a result of, for example, termination of transcription or blocking of ribosome binding to the RNA. Binding of a trigger molecule or an analog thereof can, depending on the nature of the riboswitch, reduce or prevent expression of the RNA molecule or promote or increase expression of the RNA molecule.

Also disclosed are compositions and methods for regulating expression of a naturally occurring gene or RNA that contains a lysine riboswitch by activating, deactivating or blocking the riboswitch. If the gene is essential for survival of a cell or organism that harbors it, activating, deactivating or blocking the lysine riboswitch can result in death, stasis or debilitation of the cell or organism. For example, activating a naturally occurring riboswitch in a naturally occurring gene that is essential to survival of a microorganism can result in death of the microorganism (if activation of the riboswitch turns off or represses expression). This is one basis for the use of the disclosed compounds and methods for antimicrobial and antibiotic effects.

Disclosed herein is a method of inhibiting gene expression, the method comprising (a) bringing into contact a compound and a cell, (b) wherein the compound has the structure of Formula I:

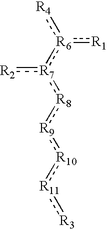

wherein R₂ and R₃ are each independently positively charged, can serve as a hydrogen bond donor, or both,

wherein R₁ is negatively charged, R₄ is negatively charged, or R₁ and R₄ are in a resonance hybrid with a net negative charge,

wherein at least one of R₁ or R₄ can be CH₂, CH₃, NH, O, O⁻, OH, S, S⁻, SH, C—R₁₄, CH—R₁₄, or N—R₁₄, wherein R₁₄ can be CH₂, CH₃, O, O⁻, OH, S, S⁻, or SH, wherein R₉ can be C, CH, CH₂, NH, O, S, C—R₅, CH—R₅, or N—R₅, wherein R₅ can be methyl, ethyl, propyl, isopropyl, cyclopropyl, butyl, tert-butyl, sec-butyl, iso-butyl, cyclobutyl, ethenyl, 3-propenyl, 1-propenyl, isopropenyl, 3-butenyl, 4-butenyl, 3-propynyl, 3-butynyl, 4-butynyl, diazirinyl, aziridinyl, urazolyl, azetidinyl, pyrazolidinyl, imidazolidinyl, oxazolidinyl, isoxazolinyl, isoxazolyl, thiazolidinyl, isothiazolyl, isothiazolinyl, oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, or piperidin-2-onyl (valerolactam),

wherein R₂ is NH₂⁺, NH₃⁺, OH, SH, NOH, NHNH₂, NHNH₃⁺, CO₂H, SO₂OH, B(OH)₂, or imidazolium,

wherein R₃ can be N, NH, NH₂⁺, NH₃⁺, O, OH, S, SH, C—R₁₃, CH—R₁₃, N—R₁₃, NH—R₁₃, O—R₁₃, or S—R₁₃, wherein R₁₃ is NH₂⁺, NH₃⁺, CO₂H, B(OH)₂, CH(NH₂)₂, C(NH₂)₂⁺, CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present,

wherein R₇ can have an S configuration based on a priority of R₂>R₆>R₈,

wherein R₆, R₇, R₈, R₁₀, and R₁₁ each independently can be C, CH, CH₂, N, NH, O, or S,

wherein [symbol] can each independently represent a single or double bond, and

wherein the compound is not lysine, and wherein the cell comprises a gene encoding an RNA comprising a lysine-responsive riboswitch, wherein the compound inhibits expression of the gene by binding to the lysine-responsive riboswitch.

R₃ can be positively charged and can serve as a hydrogen bond donor. R₅ can be uncharged. R₉ can be C, O, or S. The pKₐ of R₃ can be 7 or higher. R₁₃ can be positively charged, and can serve as a hydrogen bond donor, or both.

In one example, R₆, R₇, R₈, R₉, R₁₀ and R₁₁ are not all simultaneously C, CH, or CH₂.

In another example, R₁, R₂, R₃, R₄ and R₉ are not simultaneously O, NH₃⁺, NH₃⁺, O and S, respectively. Furthermore, in another example, R₁, R₂, R₃, and R₄ are not simultaneously O, H, NH₃⁺, and O, respectively. In another example, R₁, R₂, R₃, R₄ and R₉ are not simultaneously CO₂⁻, NH₃⁺, NH₃⁺, and H, respectively. In a further example, R₁, R₂, R₃, R₄ and R₁₁ are not simultaneously O, NH₃⁺, NH₃⁺, O and C—CO₂⁻, respectively. In a further example, R₁, R₂, R₃, and R₄ are not simultaneously NHOH, NH₃⁺, NH₃⁺, O and S, respectively.

In one example, R₉ can be NH, O, S, C—R₅, CH—R₅, or N—R₅, wherein R₅ is methyl, ethyl, propyl, isopropyl, cyclopropyl, butyl, tert-butyl, sec-butyl, iso-butyl, cyclobutyl, ethenyl, 3-propenyl, 1-propenyl, isopropenyl, 3-butenyl, 4-butenyl, 3-propynyl, 3-butynyl, 4-butynyl, diazirinyl, aziridinyl, urazolyl, azetidinyl, pyrazolidinyl, imidazolidinyl, oxazolidinyl, isoxazolinyl, isoxazolyl, thiazolidinyl, isothiazolyl, isothiazolinyl, oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, or piperidin-2-onyl (valerolactam).

The compound wherein R₂ is NH₂⁺, OH, SH, NOH, NHNH₂, NHNH₃⁺, CO₂H, SO₂OH, B(OH)₂, or imidazolium.

In another example, R₃ can be N, NH, NH₂⁺, O, OH, S, SH, C—R₁₃, CH—R₁₃, N—R₁₃, NH—R₁₃, O—R₁₃, or S—R₁₃, wherein R₁₃ is NH₂⁺, NH₃⁺, CO₂H, B(OH)₂, CH(NH₂)₂, C(NH₂)₂⁺, CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present.

In a further example, R₁₀ can be N, NH, O, or S. In a further example, R₇ can be CH.

The cell can be identified as being in need of inhibited gene expression. The cell can be a bacterial cell, for example, and the compound can kill or inhibit the growth of the bacterial cell. The compound and the cell can be brought into contact by administering the compound to a subject. In one example, the compound is not a substrate for enzymes of the subject that have lysine as a substrate. The compound can also not be a substrate for enzymes of the subject that alter lysine. The compound can also not be a substrate for enzymes of the subject that metabolize lysine. The compound can also not be a substrate for enzymes of the subject that catabolize lysine. The cell can be a bacterial cell in the subject, wherein the compound kills or inhibits the growth of the bacterial cell.

Disclosed herein is a compound having the structure of Formula I:

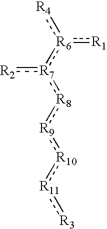

wherein R₂ and R₃ are each independently positively charged, can serve as a hydrogen bond donor, or both,

wherein R₁ is negatively charged, R₄ is negatively charged, or R₁ and R₄ are in a resonance hybrid with a net negative charge,

wherein at least one of R₁ or R₄ can be CH₂, CH₃, NH, O, O⁻, OH, S, S⁻, SH, C—R₁₄, CH—R₁₄, or N—R₁₄, wherein R₁₄ can be CH₂, CH₃, O, O⁻, OH, S, S⁻, or SH,

wherein R₉ can be C, CH, CH₂, NH, O, S, C—R₅, CH—R₅, or N—R₅, wherein R₅ can be methyl, ethyl, propyl, isopropyl, cyclopropyl, butyl, tert-butyl, sec-butyl, iso-butyl, cyclobutyl, ethenyl, 3-propenyl, 1-propenyl, isopropenyl, 3-butenyl, 4-butenyl, 3-propynyl, 3-butynyl, 4-butynyl, diazirinyl, aziridinyl, urazolyl, azetidinyl, pyrazolidinyl, imidazolidinyl, oxazolidinyl, isoxazolinyl, isoxazolyl, thiazolidinyl, isothiazolyl, isothiazolinyl, oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, or piperidin-2-onyl (valerolactam),

wherein R₂ is NH₂⁺, NH₃⁺, OH, SH, NOH, NHNH₂, NHNH₃⁺, CO₂H, SO₂OH, B(OH)₂, or imidazolium,

wherein R₃ can be N, NH, NH₂⁺, NH₃⁺, O, OH, S, SH, C—R₁₃, CH—R₁₃, N—R₁₃, NH—R₁₃, O—R₁₃, or S—R₁₃, wherein R₁₃ is NH₂⁺, NH₃⁺, CO₂H, B(OH)₂, CH(NH₂)₂, C(NH₂)₂⁺, CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4-diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present,

wherein R₇ can have an S configuration based on a priority of R₂>R₆>R₈,

wherein R₆, R₇, R₈, R₁₀, and R₁₁ each independently can be C, CH, CH₂, N, NH, O, or S,

wherein [symbol] can each independently represent a single or double bond, and

wherein the compound is not lysine.

R₃ can be positively charged and can serve as a hydrogen bond donor. R₅ can be uncharged. R₉ can be C, O, or S. The pKₐ of R₃ can be 7 or higher. R₁₃ can be positively charged, and can serve as a hydrogen bond donor, or both.

In one example, R₆, R₇, R₈, R₉, R₁₀ and R₁₁ are not all simultaneously C, CH, or CH₂.

In another example, R₁, R₂, R₃, R₄ and R₉ are not simultaneously O, NH₃⁺, NH₃⁺, O and S, respectively. Furthermore, in another example, R₁, R₂, R₃, and R₄ are not simultaneously O, H, NH₃⁺, and O, respectively. In another example, R₁, R₂, R₃, R₄ and R₉ are not simultaneously CO₂⁻, NH₃⁺, NH₃⁺, and H, respectively. In a further example, R₁, R₂, R₃, R₄ and R₁₁ are not simultaneously O, NH₃⁺, NH₃⁺, O and C—CO₂⁻, respectively. In a further example, R₁, R₂, R₃, and R₄ are not simultaneously NHOH, NH₃⁺, NH₃⁺, O and S, respectively.

In one example, R₉ can be NH, O, S, C—R₅, CH—R₅, or N—R₅, wherein R₅ is methyl, ethyl, propyl, isopropyl, cyclopropyl, butyl, tert-butyl, sec-butyl, iso-butyl, cyclobutyl, ethenyl, 3-propenyl, 1-propenyl, isopropenyl, 3-butenyl, 4-butenyl, 3-propynyl, 3-butynyl, 4-butynyl, diazirinyl, aziridinyl, urazolyl, azetidinyl, pyrazolidinyl, imidazolidinyl, oxazolidinyl, isoxazolinyl, isoxazolyl, thiazolidinyl, isothiazolyl, isothiazolinyl, oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, or piperidin-2-onyl (valerolactam).

The compound wherein R₂ is NH₂⁺, OH, SH, NOH, NHNH₂, NHNH₃⁺, CO₂H, SO₂OH, B(OH)₂, or imidazolium.

In another example, R₃ can be N, NH, NH₂⁺, O, OH, S, SH, C—R₁₃, CH—R₁₃, N—R₁₃, NH—R₁₃, O—R₁₃, or S—R₁₃, wherein R₁₃ is NH₂⁺, NH₃⁺, CO₂H, B(OH)₂, CH(NH₂)₂, C(NH₂)₂⁺, CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present.

In a further example, R₁₀ can be N, NH, O, or S. In a further example, R₇ can be CH.

Further disclosed is a composition comprising the compound described above and a regulatable gene expression construct comprising a nucleic acid molecule encoding an RNA comprising a lysine riboswitch operably linked to a coding region, wherein the lysine riboswitch regulates expression of the RNA, wherein the lysine riboswitch and coding region are heterologous. The lysine riboswitch can produce a signal when activated by the compound. For example, the riboswitch can change conformation when activated by the compound, and the change in conformation can produce a signal via a conformation dependent label. Furthermore, the riboswitch can change conformation when activated by the compound, wherein the change in conformation causes a change in expression of the coding region linked to the riboswitch, wherein the change in expression produces a signal. The signal can be produced by a reporter protein expressed from the coding region linked to the riboswitch.

Also disclosed is a method comprising: (a) testing the compound as described above for inhibition of gene expression of a gene encoding an RNA comprising a lysine riboswitch, wherein the inhibition is via the lysine riboswitch, and (b) inhibiting gene expression by bringing into contact a cell and a compound that inhibited gene expression in step (a), wherein the cell comprises a gene encoding an RNA comprising the lysine riboswitch, wherein the compound inhibits expression of the gene by binding to the lysine riboswitch.

Further disclosed is a method of inhibiting the growth of and/or killing bacteria, comprising contacting the bacteria with a compound disclosed above. Disclosed herein is also a method of inhibiting growth of a cell, such as a bacterial cell, that is in a subject, the method comprising administering an effective amount of a compound as disclosed herein to the subject. This can result in the compound being brought into contact with the cell. The subject can have, for example, a bacterial infection, and the bacterial cells can be the cells to be inhibited by the compound. The bacteria can be any bacteria. Bacterial growth can also be inhibited in any context in which bacteria are found. For example, bacterial growth in fluids, biofilms, and on surfaces can be inhibited. The compounds disclosed herein can be administered or used in combination with any other compound or composition. For example, the disclosed compounds can be administered or used in combination with another antimicrobial compound.

Also disclosed are compositions and methods for selecting and identifying compounds that can activate, deactivate or block a riboswitch. Activation of a riboswitch refers to the change in state of the riboswitch upon binding of a trigger molecule. A riboswitch can be activated by compounds other than the trigger molecule and in ways other than binding of a trigger molecule. The term trigger molecule is used herein to refer to molecules and compounds that can activate a riboswitch. This includes the natural or normal trigger molecule for the riboswitch and other compounds that can activate the riboswitch. Natural or normal trigger molecules are the trigger molecule for a given riboswitch in nature or, in the case of some non-natural riboswitches, the trigger molecule for which the riboswitch was designed or with which the riboswitch was selected (as in, for example, in vitro selection or in vitro evolution techniques). Non-natural trigger molecules can be referred to as non-natural trigger molecules.

Deactivation of a riboswitch refers to the change in state of the riboswitch when the trigger molecule is not bound. A riboswitch can be deactivated by binding of compounds other than the trigger molecule and in ways other than removal of the trigger molecule. Blocking of a riboswitch refers to a condition or state of the riboswitch where the presence of the trigger molecule does not activate the riboswitch. Activation of a riboswitch can be assessed in any suitable manner. For example, the riboswitch can be linked to a reporter RNA and expression, expression level, or change in expression level of the reporter RNA can be measured in the presence and absence of the test compound. As another example, the riboswitch can include a conformation dependent label, the signal from which changes depending on the activation state of the riboswitch. Such a riboswitch preferably uses an aptamer domain from or derived from a naturally occurring riboswitch. As can be seen, assessment of activation of a riboswitch can be performed with the use of a control assay or measurement or without the use of a control assay or measurement. Methods for identifying compounds that deactivate a riboswitch can be performed in analogous ways.

Also disclosed are compounds made by identifying a compound that activates, deactivates or blocks a riboswitch and manufacturing the identified compound. This can be accomplished by, for example, combining compound identification methods as disclosed elsewhere herein with methods for manufacturing the identified compounds. For example, compounds can be made by bringing into contact a test compound and a riboswitch, assessing activation of the riboswitch, and, if the riboswitch is activated by the test compound, manufacturing the test compound that activates the riboswitch as the compound.

Also disclosed are compounds made by checking activation, deactivation or blocking of a riboswitch by a compound and manufacturing the checked compound. This can be accomplished by, for example, combining compound activation, deactivation or blocking assessment methods as disclosed elsewhere herein with methods for manufacturing the checked compounds. For example, compounds can be made by bringing into contact a test compound and a riboswitch, assessing activation of the riboswitch, and, if the riboswitch is activated by the test compound, manufacturing the test compound that activates the riboswitch as the compound. Checking compounds for their ability to activate, deactivate or block a riboswitch refers to both identification of compounds previously unknown to activate, deactivate or block a riboswitch and to assessing the ability of a compound to activate, deactivate or block a riboswitch where the compound was already known to activate, deactivate or block the riboswitch.

Disclosed herein is also a method of inhibiting growth of a cell, such as a bacterial cell, that is in a subject, the method comprising administering an effective amount of a compound as disclosed herein to the subject. This can result in the compound being brought into contact with the cell. The subject can have, for example, a bacterial infection, and the bacterial cells can be the cells to be inhibited by the compound. The bacteria can be any bacteria, such as bacteria from the genus Bacillus, Actinobacillus, Clostridium, Desulfitobacterium, Enterococcus, Erwinia, Escherichia, Exiguobacterium, Fusobacterium, Geobacillus, Haemophilus, Idiomarina, Lactobacillus, Lactococcus, Leuconostoc, Listeria, Moorella, Oceanobacillus, Oenococcus, Pasteurella, Pediococcus, Shewanella, Shigella, Solibacter, Staphylococcus, Thermoanaerobacter, Thermotoga, and Vibrio, for example. Bacterial growth can also be inhibited in any context in which bacteria are found. For example, bacterial growth in fluids, biofilms, and on surfaces can be inhibited. The compounds disclosed herein can be administered or used in combination with any other compound or composition. For example, the disclosed compounds can be administered or used in combination with another antimicrobial compound.

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows the structure and function of the lysC riboswitch from B. subtilis. (a) The sequence and secondary structure model of the repressed-state lysC 5′-UTR from B. subtilis. Certain nucleotides are conserved in at least 90% of the representatives identified by bioinformatics. The putative antiterminator hairpin that forms in the absence of ligand is also shown. An additional 63 nucleotides reside between nucleotide 268 and the lysC start codon. A 179-nucleotide construct (179 lysC) spanning nucleotides 27 through 205 (bracketed) was used to determine ligand binding affinities. Nucleotides where spontaneous cleavage activity changes upon ligand binding are encircled (regions A, B, and C) and correspond to the bands identified in FIG. 2b. (b) The lysine biosynthesis pathway in B. subtilis. The name of the gene that codes for the enzyme or transporter at each step is indicated adjacent to a solid arrow. The expression of aspartokinase II and a lysine-specific importer (lysC and yvsH, boxed) is regulated by a lysine riboswitch in the 5′-UTR of each gene. IUPAC numbering for each carbon atom of lysine is shown. The lysine analog L-aminoethylcysteine (AEC, boxed), which differs from lysine in that C4 is replaced by sulfur, is also depicted.

FIG. 2 shows molecular recognition by a lysine riboswitch receptor. (a) Chemical structures of lysine analogs examined in this study for binding to 179 lysC. The expected protonation states under the conditions used for in-line probing are shown for each functional group. Shaded regions highlight the functional groups that differ from lysine. K_{D} values for the interaction of each analog with 179 lysC are the average of two independent repeats yielding the standard deviation shown. (b) Representative denaturing polyacrylamide gel separating products generated by in-line probing analysis of 179 lysC with 2. Concentrations of L-4-oxalysine (2) used ranged from 1 nM to 6 mM. (NR) denotes untreated, full-length RNA, and (−) represents the reaction in the absence of any added compound. The length of each product band was determined by comparison to a partial digest with RNase T1 (T1) and a partial digest with alkalai (⁻OH). Numbered bands correspond to selected products of RNAse T1 digestion (G-specific cleavage). (c) Plot depicting the normalized fraction of RNA cleaved at regions A, B, and C versus the concentration of either lysine or L-4-oxalysine (2). The curves indicate the best fit of the data to an equation for a two-state binding model (Example 1). (d) Schematic representation of the molecular recognition characteristics of the lysine riboswitch.

FIG. 3 shows lysine derivatives that inhibit bacterial growth and repress gene expression. (a) The growth of B. subtilis in a chemically-defined minimal media (Example 1) was monitored in the presence of 100 μM of each lysine derivative (compounds are numbered as in FIG. 2) or in the absence of any added compound (−) by measuring absorbance of the culture at 595 nm. Growth was not significantly inhibited by the compounds that are not shown. (b) Plot of bacterial growth in the presence of 100 μM of each lysine analog after 6 h, normalized to the growth in the absence of added compound (−). Circles highlight lysine analogs that bind the riboswitch with a K_{D} below 13 μM. (c) The minimum concentration of each compound required to completely inhibit growth (MIC) over 24 h. Also shown is the expression of a β-galactosidase gene fused to a second copy of the lysC riboswitch in a wild-type B. subtilis strain after growing for 3 h in the presence of 5 mM of each indicated derivative (Miller units). The compounds highlighted in grey bind the riboswitch, fully inhibit growth, and repress gene expression. (d) β-galactosidase expression as described in c as a function of increasing concentrations of lysine, 1, or 2 from 0.3 μM to 1 mM. Relative expression denotes the Miller units at each concentration relative to the Miller units in the absence of any added compound. The arrows indicate the MIC of 1 and 2.

FIG. 4 shows mutations within the lysine riboswitch confer resistance to lysine derivatives and deregulate the lysine riboswitch. (a) Nucleotide changes in the M1 and M2 mutants identified in the B. subtilis lysC riboswitch are boxed. Changes in the in-line probing pattern caused by each mutation (shown in c) are encircled. (b) The MIC values are given for each compound toward a wild-type, M1, or M2 strain of B. subtilis. K_{D} values are for the interaction of the indicated compound with the 179-nucleotide receptor domain of the wild-type, M1 or M2 riboswitches at 37° C., and Miller units indicate the expression of a β-galactosidase gene controlled by a lysine riboswitch with no mutation or with the M1 or M2 mutation. In each case, the reporter gene was expressed in a wild-type B. subtilis strain while growing for 3 h in the presence of the indicated lysine derivative at 5 mM (WT) or 1 mM (M1 and M2). (c) In-line probing analysis of the 179-nucleotide lysC receptor domain with either the M1 or M2 mutation at the indicated temperatures. The changes in the spontaneous cleavage pattern induced by each mutation are highlighted (additional details are as described in the legend to FIG. 2b). (d) In vitro transcription analysis of the DNA templates corresponding to the wild-type, M1, or M2 lysine riboswitches. Reactions were conducted with E. coli RNA polymerase holoenzyme in the presence (+) or absence (−) of 10 mM lysine as indicated for each lane. Below each lane is noted the percentage of transcription termination (T) at the expected site (FIG. 1a) relative to the total amount of terminated plus full length (FL) RNA. (e) In vitro transcription analysis of the lysine riboswitch as a function of an increasing concentration of the indicated compound. The apparent concentration at which termination efficiency is half-maximally attained (T₅₀) by lysine is depicted with a dashed line for the WT or M2 riboswitch.

FIG. 5 shows the consensus sequence and secondary structure for the lysine riboswitch, determined by comparing the sequences of all known examples of the lysine riboswitch.

FIG. 6 shows the pathways for lysine biosynthesis and import in bacteria. Escherichia coli gene names are used throughout, with the exception of the underlined gene names, which are found in Bacillus subtilis, and the names of the putative lysine transporter genes (boxed). Most bacterial species convert tetrahydrodipicolinate to L,L-diaminopimelate via two N-succinyl intermediates, catalyzed by the products of the dapD, dapC, and dapE genes in E. coli. Some species, including B. subtilis, accomplish this conversion via N-acetyl intermediates, catalyzed by the products of the dapD, patA, and ykuR genes. In addition to synthesizing lysine, many bacteria also import lysine from their environment. The well-characterized lysine-specific importer, coded by lysP in E. coli, several Gram negative and Gram positive species, In addition, three other putative lysine transporters were recently identified by comparative analysis of genes regulated by lysine riboswitches. The yvsH gene of B. subtilis codes for a putative lysine transporter with high sequence similarity to the APA basic amino acid/polyamine antiporter family. The lys W class of genes, found in Vibrio and Shewanella species, code for a putative transporter with high sequence similarity to the NhaC Na+:H+ antiporter superfamily. The lysXY class of putative lysine transporters has high sequence similarity to an ATP-dependent transport system for other amino acids.

FIG. 7 shows growth of B. subtilis lysine auxotroph strain 1A40 upon supplementation of minimal media with various compounds as indicated. Both compound 3 (see FIG. 2 for compound identities) and lysine support growth in a chemically-defined minimal media (Example 1). Growth was established by measuring the absorbance at 600 nm after 3 h in the presence of 1 mM of the compounds indicated or in the absence of added compound (−).

### DETAILED DESCRIPTION OF THE INVENTION

The disclosed methods and compositions can be understood more readily by reference to the following detailed description of particular embodiments and the Examples included therein and to the Figures and their previous and following description.

Messenger RNAs are typically thought of as passive carriers of genetic information that are acted upon by protein- or small RNA-regulatory factors and by ribosomes during the process of translation. It was discovered that certain mRNAs carry natural aptamer domains and that binding of specific metabolites directly to these RNA domains leads to modulation of gene expression. Natural riboswitches exhibit two surprising functions that are not typically associated with natural RNAs. First, the mRNA element can adopt distinct structural states wherein one structure serves as a precise binding pocket for its target metabolite. Second, the metabolite-induced allosteric interconversion between structural states causes a change in the level of gene expression by one of several distinct mechanisms. Riboswitches typically can be dissected into two separate domains: one that selectively binds the target (aptamer domain) and another that influences genetic control (expression platform). It is the dynamic interplay between these two domains that results in metabolite-dependent allosteric control of gene expression.

Distinct classes of riboswitches have been identified and are shown to selectively recognize activating compounds (referred to herein as trigger molecules). For example, coenzyme B₁₂, glycine, thiamine pyrophosphate (TPP), and flavin mononucleotide (FMN) activate riboswitches present in genes encoding key enzymes in metabolic or transport pathways of these compounds. The aptamer domain of each riboswitch class conforms to a highly conserved consensus sequence and structure. Thus, sequence homology searches can be used to identify related riboswitch domains. Riboswitch domains have been discovered in various organisms from bacteria, archaea, and eukarya.

Lysine riboswitches are bacterial RNA structures that sense the concentration of lysine and regulate the expression of lysine biosynthesis and transport genes. Members of this riboswitch class are found in the 5′-untranslated region (5′-UTR) of messenger RNAs, where they form highly selective receptors for lysine. Lysine binding to the receptor stabilizes an mRNA tertiary structure that, in most cases, causes transcription termination before the adjacent open reading frame can be expressed. A lysine riboswitch can be used for antibacterial therapy by designing compounds that bind the riboswitch and suppress lysine biosynthesis and transport genes. Lysine analogs that bind to riboswitches and thereby inhibit bacterial growth have been identified, and their mechanism of action elucidated (Example 1).

### A. General Organization of Riboswitch RNAs

Bacterial riboswitch RNAs are genetic control elements that are located primarily within the 5′-untranslated region (5′-UTR) of the main coding region of a particular mRNA. Structural probing studies (discussed further below) reveal that riboswitch elements are generally composed of two domains: a natural aptamer (T. Hermann, D. J. Patel, Science 2000, 287, 820; L. Gold, et al., Annual Review of Biochemistry 1995, 64, 763) that serves as the ligand-binding domain, and an ‘expression platform’ that interfaces with RNA elements that are involved in gene expression (e.g. Shine-Dalgarno (SD) elements; transcription terminator stems). These conclusions are drawn from the observation that aptamer domains synthesized in vitro bind the appropriate ligand in the absence of the expression platform (see Examples 2, 3 and 6 of U.S. Application Publication No. 2005-0053951). Moreover, structural probing investigations suggest that the aptamer domain of most riboswitches adopts a particular secondary- and tertiary-structure fold when examined independently, that is essentially identical to the aptamer structure when examined in the context of the entire 5′ leader RNA. This indicates that, in many cases, the aptamer domain is a modular unit that folds independently of the expression platform (see Examples 2, 3 and 6 of U.S. Application Publication No. 2005-0053951).

Ultimately, the ligand-bound or unbound status of the aptamer domain is interpreted through the expression platform, which is responsible for exerting an influence upon gene expression. The view of a riboswitch as a modular element is further supported by the fact that aptamer domains are highly conserved amongst various organisms (and even between kingdoms as is observed for the TPP riboswitch), (N. Sudarsan, et al., RNA 2003, 9, 644) whereas the expression platform varies in sequence, structure, and in the mechanism by which expression of the appended open reading frame is controlled. For example, ligand binding to the TPP riboswitch of the tenA mRNA of B. subtilis causes transcription termination (A. S. Mironov, et al., Cell 2002, 111, 747). This expression platform is distinct in sequence and structure compared to the expression platform of the TPP riboswitch in the thiM mRNA from E. coli, wherein TPP binding causes inhibition of translation by a SD blocking mechanism (see Example 2 of U.S. Application Publication No. 2005-0053951). The TPP aptamer domain is easily recognizable and of near identical functional character between these two transcriptional units, but the genetic control mechanisms and the expression platforms that carry them out are very different.

Aptamer domains for riboswitch RNAs typically range from ˜70 to 170 nt in length (FIG. 11 of U.S. Application Publication No. 2005-0053951). This observation was somewhat unexpected given that in vitro evolution experiments identified a wide variety of small molecule-binding aptamers, which are considerably shorter in length and structural intricacy (T. Hermann, D. J. Patel, Science 2000, 287, 820; L. Gold, et al., Annual Review of Biochemistry 1995, 64, 763; M. Famulok, Current Opinion in Structural Biology 1999, 9, 324). Although the reasons for the substantial increase in complexity and information content of the natural aptamer sequences relative to artificial aptamers remains to be proven, this complexity is believed required to form RNA receptors that function with high affinity and selectivity. Apparent K_{D} values for the ligand-riboswitch complexes range from low nanomolar to low micromolar. It is also worth noting that some aptamer domains, when isolated from the appended expression platform, exhibit improved affinity for the target ligand over that of the intact riboswitch. (˜10 to 100-fold) (see Example 2 of U.S. Application Publication No. 2005-0053951). Presumably, there is an energetic cost in sampling the multiple distinct RNA conformations required by a fully intact riboswitch RNA, which is reflected by a loss in ligand affinity. Since the aptamer domain must serve as a molecular switch, this might also add to the functional demands on natural aptamers that might help rationalize their more sophisticated structures.

### B. Riboswitch Regulation of Transcription Termination in Bacteria

Bacteria primarily make use of two methods for termination of transcription. Certain genes incorporate a termination signal that is dependent upon the Rho protein, (J. P. Richardson, Biochimica et Biophysica Acta 2002, 1577, 251). while others make use of Rho-independent terminators (intrinsic terminators) to destabilize the transcription elongation complex (I. Gusarov, E. Nudler, Molecular Cell 1999, 3, 495; E. Nudler, M. E. Gottesman, Genes to Cells 2002, 7, 755). The latter RNA elements are composed of a GC-rich stem-loop followed by a stretch of 6-9 uridyl residues. Intrinsic terminators are widespread throughout bacterial genomes (F. Lillo, et al., 2002, 18, 971), and are typically located at the 3′-termini of genes or operons. Interestingly, an increasing number of examples are being observed for intrinsic terminators located within 5′-UTRs.

Amongst the wide variety of genetic regulatory strategies employed by bacteria there is a growing class of examples wherein RNA polymerase responds to a termination signal within the 5′-UTR in a regulated fashion (T. M. Henkin, Current Opinion in Microbiology 2000, 3, 149). During certain conditions the RNA polymerase complex is directed by external signals either to perceive or to ignore the termination signal. Although transcription initiation might occur without regulation, control over mRNA synthesis (and of gene expression) is ultimately dictated by regulation of the intrinsic terminator. Presumably, one of at least two mutually exclusive mRNA conformations results in the formation or disruption of the RNA structure that signals transcription termination. A trans-acting factor, which in some instances is a RNA (F. J. Grundy, et al., Proceedings of the National Academy of Sciences of the United States of America 2002, 99, 11121; T. M. Henkin, C. Yanofsky, Bioessays 2002, 24, 700) and in others is a protein (J. Stulke, Archives of Microbiology 2002, 177, 433), is generally required for receiving a particular intracellular signal and subsequently stabilizing one of the RNA conformations. Riboswitches offer a direct link between RNA structure modulation and the metabolite signals that are interpreted by the genetic control machinery.

Most clinical antibacterial compounds target one of only four cellular processes (Wolfson 2006). Since bacteria have well developed resistance mechanisms to protect these processes (D′Costa 2006), it is useful to discover new targets that are vulnerable to drug intervention. One type of vulnerable process is the regulation of gene expression by riboswitches (Winkler 2005). Typically found in the 5′-UTRs of certain bacterial mRNAs, members of each known riboswitch class form a structured receptor (or “aptamer”) (Mandal 2004) that has evolved to bind a specific fundamental metabolite. In most cases, ligand binding regulates the expression of a gene or group of genes involved in the synthesis or transport of the bound metabolite. Because the biochemical pathways regulated by riboswitches are often essential for bacterial survival, repression of these pathways through riboswitch targeting can be lethal.

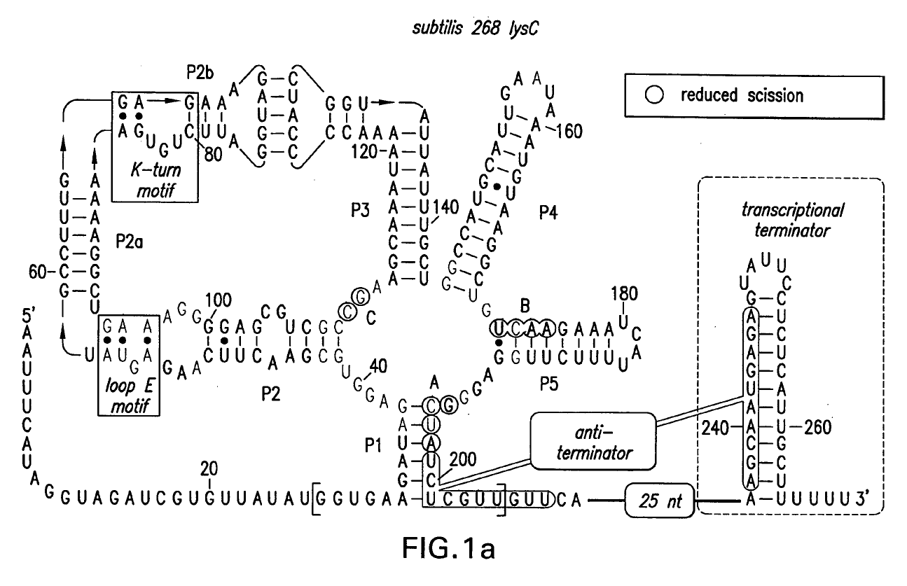

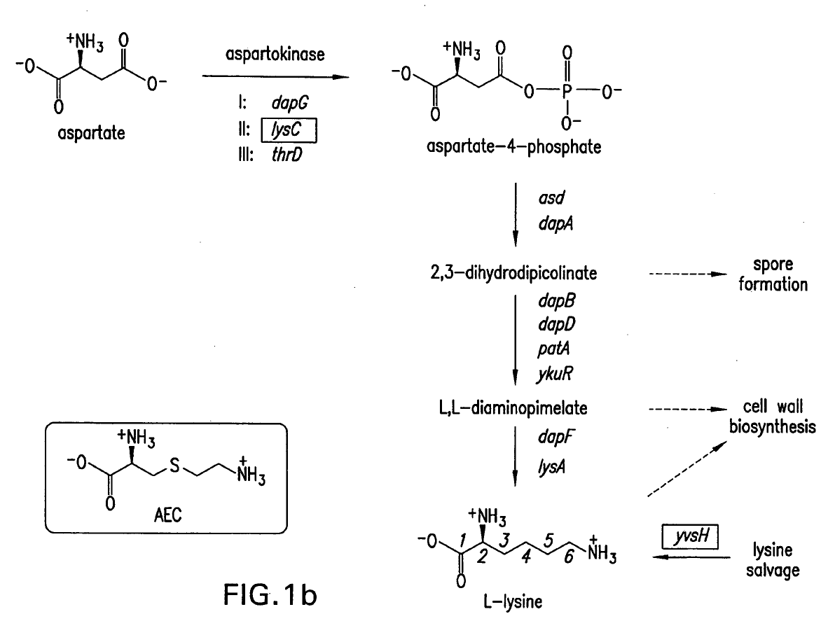

Phylogenetic sequence comparison and structural probing data revealed that, when bound to lysine, the receptor domain of a lysine riboswitch forms a secondary structure comprised of five stem-loops (P1 through P5) that radiate from a highly conserved single-stranded core (FIG. 1a; Supplementary FIG. 1) (Sudarsan 2003; Grundy 2003; Rodionov 2003). The terminal loops of stems P2 and P3 base pair with one another, and P2 also contains both a loop-E structural motif (Wimberly 1993) and a K-turn motif (Klein 2001). In most bacteria, stabilization of this structure by lysine binding permits the formation of a transcription terminator that halts RNA synthesis before the downstream open reading frame (ORF) can be transcribed (Sudarsan 2003). At subsaturating lysine concentrations, the riboswitch forms an alternate structure in which an antiterminator hairpin (FIG. 1a) precludes formation of the terminator hairpin, thus enabling normal transcription of the adjoining ORF.

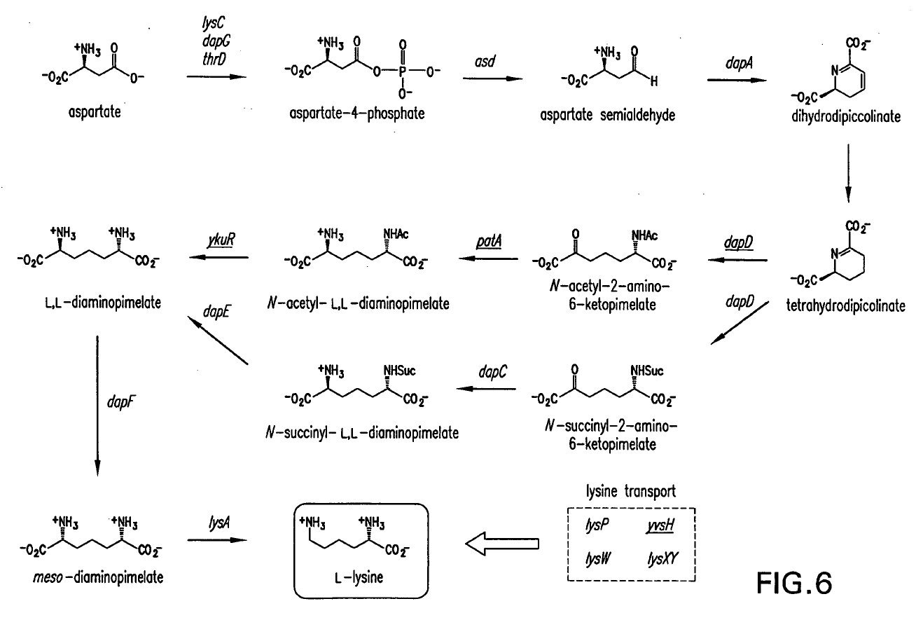

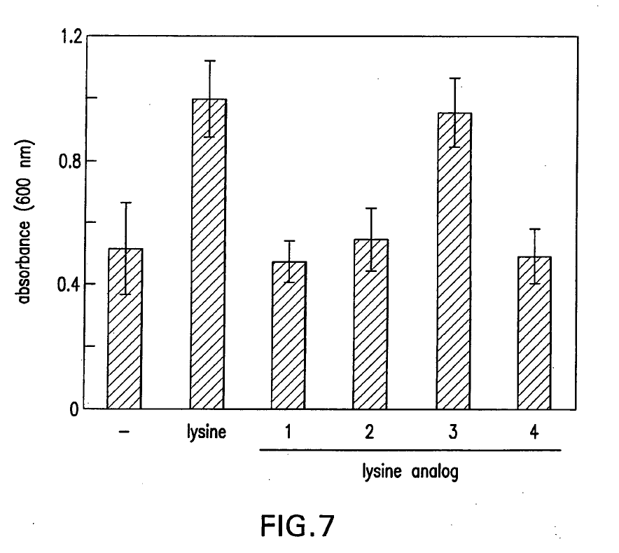

In many bacteria, including some clinically relevant pathogens (Table 1), a lysine riboswitch regulates the expression of aspartokinase II (coded by the lysC gene in Bacillus subtilis, FIG. 1b), which catalyzes the first step in lysine, threonine, and methionine biosynthesis (FIG. 6) (Sudarsan 2003; Grundy 2003; Rodionov 2003). Two lysine intermediates downstream of aspartate-4-phosphate—2,3-dihydropicolinate and L,L-diaminopimelate—are also precursors for cell wall biosynthesis and spore formation (Hutton 2003; Bugg 1994). Many bacteria also have a second copy of the riboswitch that regulates the expression of a lysine-specific importer (coded by the yvsH gene in B. subtilis) (Rodionov 2003). Compounds are disclosed herein that bind to the lysine riboswitch receptor and inhibit growth by repressing these genes, even when the bacterium is starved for lysine.

Several other antibacterial metabolite analogs function by targeting riboswitches (Sudarsan 2003; Sudarsan 2005; Woolley 1943). For example, the antibacterial thiamine analog pyrithiamine (Woolley 1943) most likely functions by targeting a thiamine pyrophosphate-binding riboswitch (Sudarsan 2005). Similarly, the antibacterial lysine analog L-aminoethylcysteine (Shiota 1958) (AEC, FIG. 1b) binds to the lysC riboswitch from B. subtilis and represses the expression of a lysC-regulated reporter gene (Sudarsan 2006). Moreover, the lysC riboswitch is mutated in B. subtilis (Lu 1991) and Escherichia coli (Patte 1998) strains resistant to AEC.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

### Materials

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference to each of various individual and collective combinations and permutation of these compounds can not be explicitly disclosed, each is specifically contemplated and described herein. For example, if a riboswitch or aptamer domain is disclosed and discussed and a number of modifications that can be made to a number of molecules including the riboswitch or aptamer domain are discussed, each and every combination and permutation of riboswitch or aptamer domain and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Riboswitches

Riboswitches are expression control elements that are part of an RNA molecule to be expressed and that change state when bound by a trigger molecule. Riboswitches typically can be dissected into two separate domains: one that selectively binds the target (aptamer domain) and another that influences genetic control (expression platform domain). It is the dynamic interplay between these two domains that results in metabolite-dependent allosteric control of gene expression. Disclosed are isolated and recombinant riboswitches, recombinant constructs containing such riboswitches, heterologous sequences operably linked to such riboswitches, and cells and transgenic organisms harboring such riboswitches, riboswitch recombinant constructs, and riboswitches operably linked to heterologous sequences. The heterologous sequences can be, for example, sequences encoding proteins or peptides of interest, including reporter proteins or peptides. Preferred riboswitches are, or are derived from, naturally occurring riboswitches.

The disclosed riboswitches, including the derivatives and recombinant forms thereof, generally can be from any source, including naturally occurring riboswitches and riboswitches designed de novo. Any such riboswitches can be used in or with the disclosed methods. However, different types of riboswitches can be defined and some such sub-types can be useful in or with particular methods (generally as described elsewhere herein). Types of riboswitches include, for example, naturally occurring riboswitches, derivatives and modified forms of naturally occurring riboswitches, chimeric riboswitches, and recombinant riboswitches. A naturally occurring riboswitch is a riboswitch having the sequence of a riboswitch as found in nature. Such a naturally occurring riboswitch can be an isolated or recombinant form of the naturally occurring riboswitch as it occurs in nature. That is, the riboswitch has the same primary structure but has been isolated or engineered in a new genetic or nucleic acid context. Chimeric riboswitches can be made up of, for example, part of a riboswitch of any or of a particular class or type of riboswitch and part of a different riboswitch of the same or of any different class or type of riboswitch; part of a riboswitch of any or of a particular class or type of riboswitch and any non-riboswitch sequence or component. Recombinant riboswitches are riboswitches that have been isolated or engineered in a new genetic or nucleic acid context.

Riboswitches can have single or multiple aptamer domains. Aptamer domains in riboswitches having multiple aptamer domains can exhibit cooperative binding of trigger molecules or can not exhibit cooperative binding of trigger molecules (that is, the aptamers need not exhibit cooperative binding). In the latter case, the aptamer domains can be said to be independent binders. Riboswitches having multiple aptamers can have one or multiple expression platform domains. For example, a riboswitch having two aptamer domains that exhibit cooperative binding of their trigger molecules can be linked to a single expression platform domain that is regulated by both aptamer domains. Riboswitches having multiple aptamers can have one or more of the aptamers joined via a linker. Where such aptamers exhibit cooperative binding of trigger molecules, the linker can be a cooperative linker.

Aptamer domains can be said to exhibit cooperative binding if they have a Hill coefficient n between x and x−1, where x is the number of aptamer domains (or the number of binding sites on the aptamer domains) that are being analyzed for cooperative binding. Thus, for example, a riboswitch having two aptamer domains (such as glycine-responsive riboswitches) can be said to exhibit cooperative binding if the riboswitch has Hill coefficient between 2 and 1. It should be understood that the value of x used depends on the number of aptamer domains being analyzed for cooperative binding, not necessarily the number of aptamer domains present in the riboswitch. This makes sense because a riboswitch can have multiple aptamer domains where only some exhibit cooperative binding.

Disclosed are chimeric riboswitches containing heterologous aptamer domains and expression platform domains. That is, chimeric riboswitches are made up an aptamer domain from one source and an expression platform domain from another source. The heterologous sources can be from, for example, different specific riboswitches, different types of riboswitches, or different classes of riboswitches. The heterologous aptamers can also come from non-riboswitch aptamers. The heterologous expression platform domains can also come from non-riboswitch sources.

Modified or derivative riboswitches can be produced using in vitro selection and evolution techniques. In general, in vitro evolution techniques as applied to riboswitches involve producing a set of variant riboswitches where part(s) of the riboswitch sequence is varied while other parts of the riboswitch are held constant. Activation, deactivation or blocking (or other functional or structural criteria) of the set of variant riboswitches can then be assessed and those variant riboswitches meeting the criteria of interest are selected for use or further rounds of evolution. Useful base riboswitches for generation of variants are the specific and consensus riboswitches disclosed herein. Consensus riboswitches can be used to inform which part(s) of a riboswitch to vary for in vitro selection and evolution.

Also disclosed are modified riboswitches with altered regulation. The regulation of a riboswitch can be altered by operably linking an aptamer domain to the expression platform domain of the riboswitch (which is a chimeric riboswitch). The aptamer domain can then mediate regulation of the riboswitch through the action of, for example, a trigger molecule for the aptamer domain. Aptamer domains can be operably linked to expression platform domains of riboswitches in any suitable manner, including, for example, by replacing the normal or natural aptamer domain of the riboswitch with the new aptamer domain. Generally, any compound or condition that can activate, deactivate or block the riboswitch from which the aptamer domain is derived can be used to activate, deactivate or block the chimeric riboswitch.

Also disclosed are inactivated riboswitches. Riboswitches can be inactivated by covalently altering the riboswitch (by, for example, crosslinking parts of the riboswitch or coupling a compound to the riboswitch). Inactivation of a riboswitch in this manner can result from, for example, an alteration that prevents the trigger molecule for the riboswitch from binding, that prevents the change in state of the riboswitch upon binding of the trigger molecule, or that prevents the expression platform domain of the riboswitch from affecting expression upon binding of the trigger molecule.

Also disclosed are biosensor riboswitches. Biosensor riboswitches are engineered riboswitches that produce a detectable signal in the presence of their cognate trigger molecule. Useful biosensor riboswitches can be triggered at or above threshold levels of the trigger molecules. Biosensor riboswitches can be designed for use in vivo or in vitro. For example, biosensor riboswitches operably linked to a reporter RNA that encodes a protein that serves as or is involved in producing a signal can be used in vivo by engineering a cell or organism to harbor a nucleic acid construct encoding the riboswitch/reporter RNA. An example of a biosensor riboswitch for use in vitro is a riboswitch that includes a conformation dependent label, the signal from which changes depending on the activation state of the riboswitch. Such a biosensor riboswitch preferably uses an aptamer domain from or derived from a naturally occurring riboswitch. Biosensor riboswitches can be used in various situations and platforms. For example, biosensor riboswitches can be used with solid supports, such as plates, chips, strips and wells.

Also disclosed are modified or derivative riboswitches that recognize new trigger molecules. New riboswitches and/or new aptamers that recognize new trigger molecules can be selected for, designed or derived from known riboswitches. This can be accomplished by, for example, producing a set of aptamer variants in a riboswitch, assessing the activation of the variant riboswitches in the presence of a compound of interest, selecting variant riboswitches that were activated (or, for example, the riboswitches that were the most highly or the most selectively activated), and repeating these steps until a variant riboswitch of a desired activity, specificity, combination of activity and specificity, or other combination of properties results.

In general, any aptamer domain can be adapted for use with any expression platform domain by designing or adapting a regulated strand in the expression platform domain to be complementary to the control strand of the aptamer domain. Alternatively, the sequence of the aptamer and control strands of an aptamer domain can be adapted so that the control strand is complementary to a functionally significant sequence in an expression platform. For example, the control strand can be adapted to be complementary to the Shine-Dalgarno sequence of an RNA such that, upon formation of a stem structure between the control strand and the SD sequence, the SD sequence becomes inaccessible to ribosomes, thus reducing or preventing translation initiation. Note that the aptamer strand would have corresponding changes in sequence to allow formation of a P1 stem in the aptamer domain. In the case of riboswitches having multiple aptamers exhibiting cooperative binding, one the P1 stem of the activating aptamer (the aptamer that interacts with the expression platform domain) need be designed to form a stem structure with the SD sequence.

As another example, a transcription terminator can be added to an RNA molecule (most conveniently in an untranslated region of the RNA) where part of the sequence of the transcription terminator is complementary to the control strand of an aptamer domain (the sequence will be the regulated strand). This will allow the control sequence of the aptamer domain to form alternative stem structures with the aptamer strand and the regulated strand, thus either forming or disrupting a transcription terminator stem upon activation or deactivation of the riboswitch. Any other expression element can be brought under the control of a riboswitch by similar design of alternative stem structures.

For transcription terminators controlled by riboswitches, the speed of transcription and spacing of the riboswitch and expression platform elements can be important for proper control. Transcription speed can be adjusted by, for example, including polymerase pausing elements (e.g., a series of uridine residues) to pause transcription and allow the riboswitch to form and sense trigger molecules.

Disclosed are regulatable gene expression constructs comprising a nucleic acid molecule encoding an RNA comprising a riboswitch operably linked to a coding region, wherein the riboswitch regulates expression of the RNA, wherein the riboswitch and coding region are heterologous. The riboswitch can comprise an aptamer domain and an expression platform domain, wherein the aptamer domain and the expression platform domain are heterologous. The riboswitch can comprise an aptamer domain and an expression platform domain, wherein the aptamer domain comprises a P1 stem, wherein the P1 stem comprises an aptamer strand and a control strand, wherein the expression platform domain comprises a regulated strand, wherein the regulated strand, the control strand, or both have been designed to form a stem structure. The riboswitch can comprise two or more aptamer domains and an expression platform domain, wherein at least one of the aptamer domains and the expression platform domain are heterologous. The riboswitch can comprise two or more aptamer domains and an expression platform domain, wherein at least one of the aptamer domains comprises a P1 stem, wherein the P1 stem comprises an aptamer strand and a control strand, wherein the expression platform domain comprises a regulated strand, wherein the regulated strand, the control strand, or both have been designed to form a stem structure.

1. Aptamer Domains

Aptamers are nucleic acid segments and structures that can bind selectively to particular compounds and classes of compounds. Riboswitches have aptamer domains that, upon binding of a trigger molecule result in a change in the state or structure of the riboswitch. In functional riboswitches, the state or structure of the expression platform domain linked to the aptamer domain changes when the trigger molecule binds to the aptamer domain. Aptamer domains of riboswitches can be derived from any source, including, for example, natural aptamer domains of riboswitches, artificial aptamers, engineered, selected, evolved or derived aptamers or aptamer domains. Aptamers in riboswitches generally have at least one portion that can interact, such as by forming a stem structure, with a portion of the linked expression platform domain. This stem structure will either form or be disrupted upon binding of the trigger molecule.

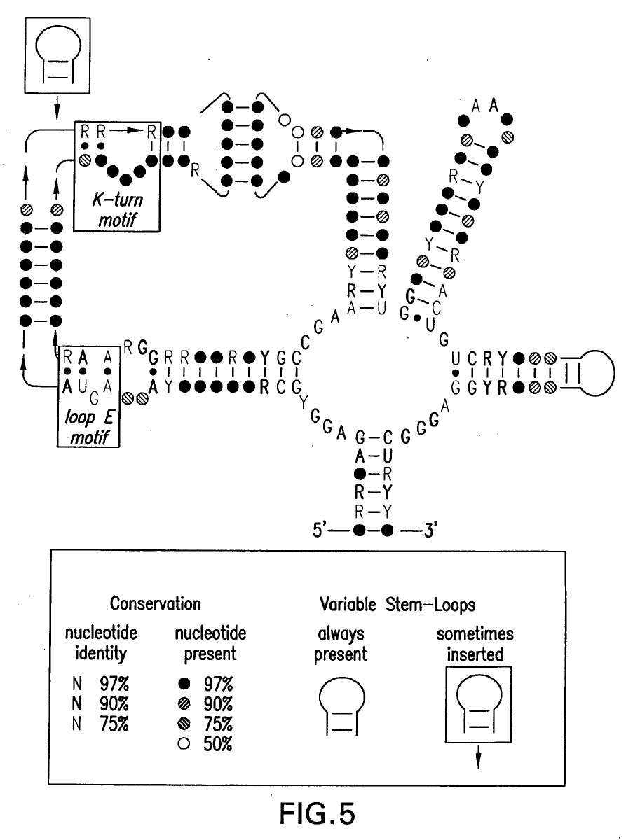

Consensus aptamer domains of a variety of natural riboswitches are shown in FIG. 11 of U.S. Application Publication No. 2005-0053951 and elsewhere herein. The consensus sequence and structure for the lysine ribozyme can be found in FIG. 5, and an example of the structure of a lysine riboswitch can be found in FIG. 1. These aptamer domains (including all of the direct variants embodied therein) can be used in riboswitches. The consensus sequences and structures indicate variations in sequence and structure. Aptamer domains that are within the indicated variations are referred to herein as direct variants. These aptamer domains can be modified to produce modified or variant aptamer domains. Conservative modifications include any change in base paired nucleotides such that the nucleotides in the pair remain complementary. Moderate modifications include changes in the length of stems or of loops (for which a length or length range is indicated) of less than or equal to 20% of the length range indicated. Loop and stem lengths are considered to be “indicated” where the consensus structure shows a stem or loop of a particular length or where a range of lengths is listed or depicted. Moderate modifications include changes in the length of stems or of loops (for which a length or length range is not indicated) of less than or equal to 40% of the length range indicated. Moderate modifications also include and functional variants of unspecified portions of the aptamer domain.

The P1 stem and its constituent strands can be modified in adapting aptamer domains for use with expression platforms and RNA molecules. Such modifications, which can be extensive, are referred to herein as P1 modifications. P1 modifications include changes to the sequence and/or length of the P1 stem of an aptamer domain. The aptamer domain is particularly useful as initial sequences for producing derived aptamer domains via in vitro selection or in vitro evolution techniques.

Aptamer domains of the disclosed riboswitches can also be used for any other purpose, and in any other context, as aptamers. For example, aptamers can be used to control ribozymes, other molecular switches, and any RNA molecule where a change in structure can affect function of the RNA.

2. Expression Platform Domains

Expression platform domains are a part of riboswitches that affect expression of the RNA molecule that contains the riboswitch. Expression platform domains generally have at least one portion that can interact, such as by forming a stem structure, with a portion of the linked aptamer domain. This stem structure will either form or be disrupted upon binding of the trigger molecule. The stem structure generally either is, or prevents formation of, an expression regulatory structure. An expression regulatory structure is a structure that allows, prevents, enhances or inhibits expression of an RNA molecule containing the structure. Examples include Shine-Dalgarno sequences, initiation codons, transcription terminators, and stability and processing signals.

### B. Trigger Molecules

Trigger molecules are molecules and compounds that can activate a riboswitch. This includes the natural or normal trigger molecule for the riboswitch and other compounds that can activate the riboswitch. Natural or normal trigger molecules are the trigger molecule for a given riboswitch in nature or, in the case of some non-natural riboswitches, the trigger molecule for which the riboswitch was designed or with which the riboswitch was selected (as in, for example, in vitro selection or in vitro evolution techniques).

### C. Compounds

Also disclosed are compounds, and compositions containing such compounds, that can activate, deactivate or block a riboswitch. Riboswitches function to control gene expression through the binding or removal of a trigger molecule. Compounds can be used to activate, deactivate or block a riboswitch. The trigger molecule for a riboswitch (as well as other activating compounds) can be used to activate a riboswitch. Compounds other than the trigger molecule generally can be used to deactivate or block a riboswitch. Riboswitches can also be deactivated by, for example, removing trigger molecules from the presence of the riboswitch. A riboswitch can be blocked by, for example, binding of an analog of the trigger molecule that does not activate the riboswitch.

Also disclosed are compounds for altering expression of an RNA molecule, or of a gene encoding an RNA molecule, where the RNA molecule includes a riboswitch. This can be accomplished by bringing a compound into contact with the RNA molecule. Riboswitches function to control gene expression through the binding or removal of a trigger molecule. Thus, subjecting an RNA molecule of interest that includes a riboswitch to conditions that activate, deactivate or block the riboswitch can be used to alter expression of the RNA. Expression can be altered as a result of, for example, termination of transcription or blocking of ribosome binding to the RNA. Binding of a trigger molecule can, depending on the nature of the riboswitch, reduce or prevent expression of the RNA molecule or promote or increase expression of the RNA molecule.

Also disclosed are compounds for regulating expression of an RNA molecule, or of a gene encoding an RNA molecule. Also disclosed are compounds for regulating expression of a naturally occurring gene or RNA that contains a riboswitch by activating, deactivating or blocking the riboswitch. If the gene is essential for survival of a cell or organism that harbors it, activating, deactivating or blocking the riboswitch can in death, stasis or debilitation of the cell or organism.

Also disclosed are compounds for regulating expression of an isolated, engineered or recombinant gene or RNA that contains a riboswitch by activating, deactivating or blocking the riboswitch. If the gene encodes a desired expression product, activating or deactivating the riboswitch can be used to induce expression of the gene and thus result in production of the expression product. If the gene encodes an inducer or repressor of gene expression or of another cellular process, activation, deactivation or blocking of the riboswitch can result in induction, repression, or de-repression of other, regulated genes or cellular processes. Many such secondary regulatory effects are known and can be adapted for use with riboswitches. An advantage of riboswitches as the primary control for such regulation is that riboswitch trigger molecules can be small, non-antigenic molecules.

Also disclosed are methods of identifying compounds that activate, deactivate or block a riboswitch. For example, compounds that activate a riboswitch can be identified by bringing into contact a test compound and a riboswitch and assessing activation of the riboswitch. If the riboswitch is activated, the test compound is identified as a compound that activates the riboswitch. Activation of a riboswitch can be assessed in any suitable manner. For example, the riboswitch can be linked to a reporter RNA and expression, expression level, or change in expression level of the reporter RNA can be measured in the presence and absence of the test compound. As another example, the riboswitch can include a conformation dependent label, the signal from which changes depending on the activation state of the riboswitch. Such a riboswitch preferably uses an aptamer domain from or derived from a naturally occurring riboswitch. As can be seen, assessment of activation of a riboswitch can be performed with the use of a control assay or measurement or without the use of a control assay or measurement. Methods for identifying compounds that deactivate a riboswitch can be performed in analogous ways.

Identification of compounds that block a riboswitch can be accomplished in any suitable manner. For example, an assay can be performed for assessing activation or deactivation of a riboswitch in the presence of a compound known to activate or deactivate the riboswitch and in the presence of a test compound. If activation or deactivation is not observed as would be observed in the absence of the test compound, then the test compound is identified as a compound that blocks activation or deactivation of the riboswitch.

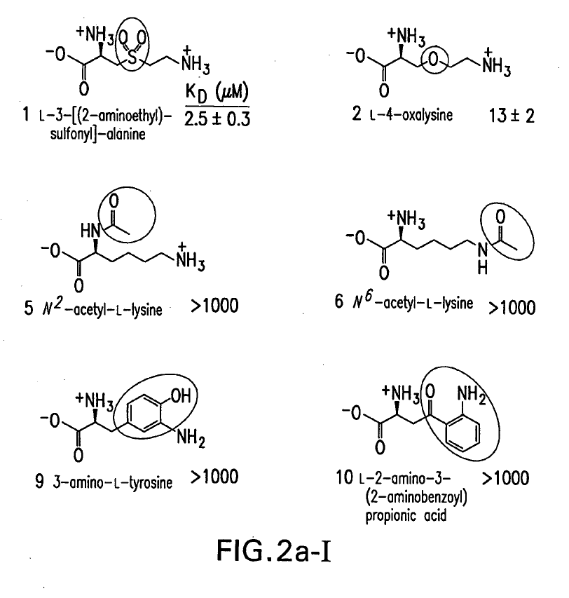

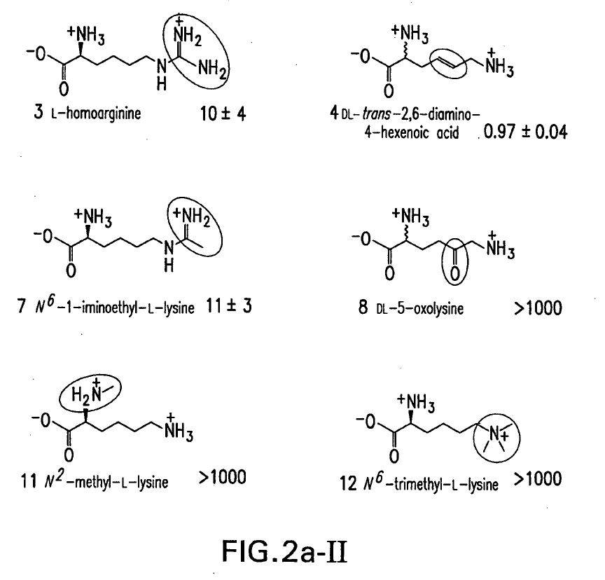

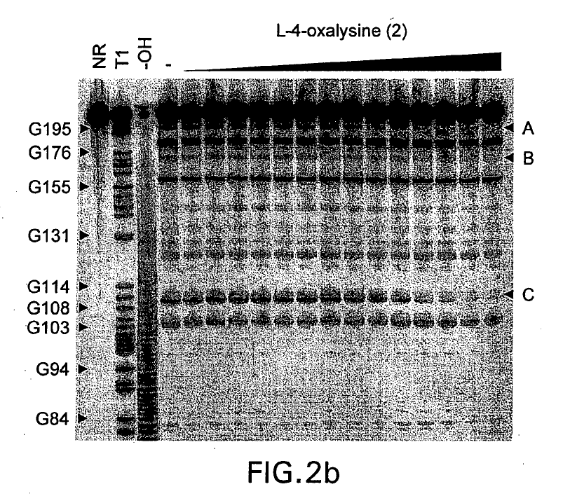

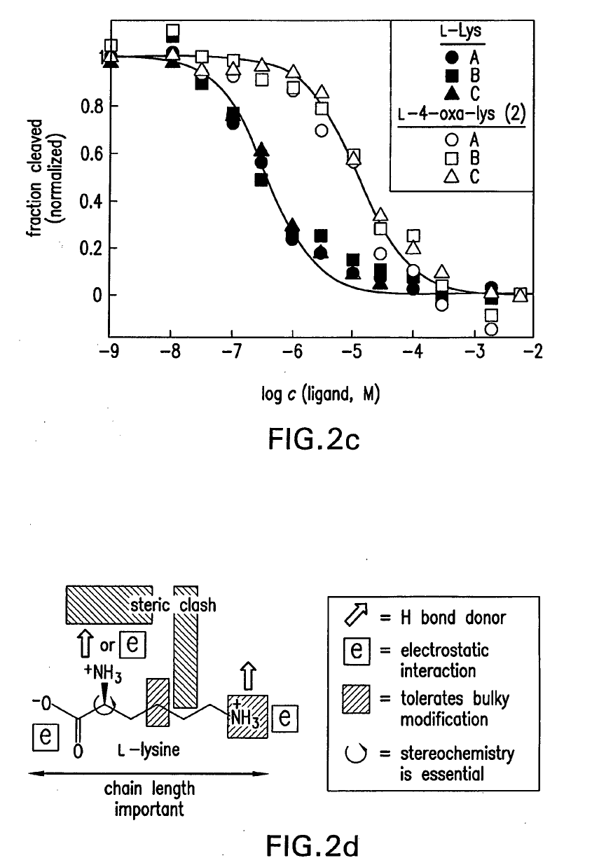

Disclosed herein are analogs that interact with the lysine riboswitch. Examples of such analogs can be found in FIG. 2. Many of the compounds synthesized and tested bind the lysine riboswitch with constants that are equal to that of lysine. The fact that appendages with highly variable chemical composition exhibit function shows that numerous variations of these chemical scaffolds can be generated and tested for function in vitro and inside cells. Specifically, further modified versions of these compounds can have improved binding to the lysine riboswitch by making new contacts to other functional groups in the RNA structure. Furthermore, modulation of bioavailability, toxicity, and synthetic ease (among other characteristics) can be tunable by making modifications in these two regions of the scaffold, as the structural model for the riboswitch shows many modifications are possible at these sites.

High-throughput screening can also be used to reveal entirely new chemical scaffolds that also bind to riboswitch RNAs either with standard or non-standard modes of molecular recognition. Since riboswitches are the first major form of natural metabolite-binding RNAs to be discovered, there has been little effort made previously to create binding assays that can be adapted for high-throughput screening. Multiple different approaches can be used to detect metabolite binding RNAs, including allosteric ribozyme assays using gel-based and chip-based detection methods, and in-line probing assays. Also disclosed are compounds made by identifying a compound that activates, deactivates or blocks a riboswitch and manufacturing the identified compound. This can be accomplished by, for example, combining compound identification methods as disclosed elsewhere herein with methods for manufacturing the identified compounds. For example, compounds can be made by bringing into contact a test compound and a riboswitch, assessing activation of the riboswitch, and, if the riboswitch is activated by the test compound, manufacturing the test compound that activates the riboswitch as the compound.

Also disclosed are compounds made by checking activation, deactivation or blocking of a riboswitch by a compound and manufacturing the checked compound. This can be accomplished by, for example, combining compound activation, deactivation or blocking assessment methods as disclosed elsewhere herein with methods for manufacturing the checked compounds. For example, compounds can be made by bringing into contact a test compound and a riboswitch, assessing activation of the riboswitch, and, if the riboswitch is activated by the test compound, manufacturing the test compound that activates the riboswitch as the compound. Checking compounds for their ability to activate, deactivate or block a riboswitch refers to both identification of compounds previously unknown to activate, deactivate or block a riboswitch and to assessing the ability of a compound to activate, deactivate or block a riboswitch where the compound was already known to activate, deactivate or block the riboswitch.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For the purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, sulfo-oxo, sulfonyl, sulfone, sulfoxide, or thiol, as described below. The term “lower alkyl” is an alkyl group with 6 or fewer carbon atoms, e.g., methyl, ethyl, propyl, isopropyl, butyl, sec-butyl, iso-butyl, tert-butyl, pentyl, hexyl, and the like.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “halogenated alkyl” is used in another, it is not meant to imply that the term “alkyl” does not also refer to specific terms such as “halogenated alkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkoxy” as used herein is an alkyl group bonded through a single, terminal ether linkage; that is, an “alkoxy” group can be defined as —OA¹ where A² is alkyl as defined above.

The term “alkenyl” as used herein is a hydrocarbon group of from 2 to 24 carbon atoms with a structural formula containing at least one carbon-carbon double bond. Asymmetric structures such as (A′A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, sulfo-oxo, sulfonyl, sulfone, sulfoxide, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” as used herein is represented by the formula —C(O)H. Throughout this specification “C(O)” is a short hand notation for C═O.

The terms “amine” or “amino” as used herein are represented by the formula NA¹A²A³, where A¹, A², and A³ can be, independently, hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “carboxylic acid” as used herein is represented by the formula —C(O)OH. A “carboxylate” as used herein is represented by the formula —C(O)O⁻.

The term “ester” as used herein is represented by the formula —OC(O)A¹ or —C(O)OA¹, where A¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula A¹OA², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula A¹C(O)A², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” as used herein refers to the halogens fluorine, chlorine, bromine, and iodine.

The term “hydroxyl” as used herein is represented by the formula —OH.

The term “sulfo-oxo” as used herein is represented by the formulas —S(O)A¹ (i.e., “sulfonyl”), A¹S(O)A² (i.e., “sulfoxide”), —S(O)₂A¹, A¹SO₂A² (i.e., “sulfone”), —OS(O)₂A¹, or —OS(O)₂OA¹, where A₁ and A² can be hydrogen, an alkyl, halogenated alkyl, alkenyl, alkynyl, aryl, heteroaryl, cycloalkyl, cycloalkenyl, heterocycloalkyl, or heterocycloalkenyl group described above. Throughout this specification “S(O)” is a short hand notation for S═O.

The term “sulfonylamino” or “sulfonamide” as used herein is represented by the formula —S(O)₂NH—.

The term “thiol” as used herein is represented by the formula —SH.

As used herein, “Rⁿ” where n is some integer can independently possess one or more of the groups listed above. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Compounds useful with lysine-responsive riboswitches (and riboswitches derived from lysine-responsive riboswitches) include compounds represented by Formula I:

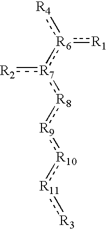

wherein R₂ and R₃ are each independently positively charged, can serve as a hydrogen bond donor, or both,

wherein R₁ is negatively charged, R₄ is negatively charged, or R₁ and R₄ are in a resonance hybrid with a net negative charge,

wherein at least one of R₁ or R₄ can be CH₂, CH₃, NH, O, O⁻, OH, S, S⁻, SH, C—R₁₄, CH—R₁₄, or N—R₁₄, wherein R₁₄ can be CH₂, CH₃, O, O⁻, OH, S, S⁻, or SH,

wherein R₉ can be C, CH, CH₂, NH, O, S, C—R₅, CH—R₅, or N—R₅, wherein R₅ can be methyl, ethyl, propyl, isopropyl, cyclopropyl, butyl, tert-butyl, sec-butyl, iso-butyl, cyclobutyl, ethenyl, 3-propenyl, 1-propenyl, isopropenyl, 3-butenyl, 4-butenyl, 3-propynyl, 3-butynyl, 4-butynyl, diazirinyl, aziridinyl, urazolyl, azetidinyl, pyrazolidinyl, imidazolidinyl, oxazolidinyl, isoxazolinyl, isoxazolyl, thiazolidinyl, isothiazolyl, isothiazolinyl, oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, or piperidin-2-onyl (valerolactam),

wherein R₂ is NH₂⁺, NH₃⁺, OH, SH, NOH, NHNH₂, NHNH₃⁺, CO₂H, SO₂OH, B(OH)₂, or imidazolium,

wherein R₃ can be N, NH, NH₂⁺, NH₃⁺, O, OH, S, SH, C—R₁₃, CH—R₁₃, N—R₁₃, NH—R₁₃, O—R₁₃, or S—R₁₃, wherein R₁₃ is NH₂⁺, NH₃⁺, CO₂H, B(OH)₂, CH(NH₂)₂, C(NH₂)₂⁺, CNH₂NH₃⁺, C(NH₃)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present,

wherein R₇ can have an S configuration based on a priority of R₂>R₆>R₈,

wherein R₆, R₇, R₈, R₁₀, and R₁₁ each independently can be C, CH, CH₂, N, NH, O, or S,

wherein [symbol] can each independently represent a single or double bond, and

wherein the compound is not lysine.

R₃ can be positively charged and can serve as a hydrogen bond donor. R₅ can be uncharged. R₉ can be C, O, or S. The pKₐ of R₃ can be 7 or higher. R₁₃ can be positively charged, and can serve as a hydrogen bond donor, or both.

In one example, R₆, R₇, R₈, R₉, R₁₀ and R₁₁ are not all simultaneously C, CH, or CH₂.

In another example, R₁, R₂, R₃, R₄ and R₉ are not simultaneously O, NH₃⁺, NH₃⁺, O and S, respectively. Furthermore, in another example, R₁, R₂, R₃, and R₄ are not simultaneously O, H, NH₃⁺, and O, respectively. In another example, R₁, R₂, R₃, R₄ and R₉ are not simultaneously CO₂⁻, NH₃⁺, NH₃⁺, and H, respectively. In a further example, R₁, R₂, R₃, R₄ and R₁₁ are not simultaneously O, NH₃⁺, NH₃⁺, O and C—CO₂⁻, respectively. In a further example, R₁, R₂, R₃, and R₄ are not simultaneously NHOH, NH₃⁺, NH₃⁺, O and S, respectively.

In one example, R₉ can be NH, O, S, C—R₅, CH—R₅, or N—R₅, wherein R₅ is methyl, ethyl, propyl, isopropyl, cyclopropyl, butyl, tert-butyl, sec-butyl, iso-butyl, cyclobutyl, ethenyl, 3-propenyl, 1-propenyl, isopropenyl, 3-butenyl, 4-butenyl, 3-propynyl, 3-butynyl, 4-butynyl, diazirinyl, aziridinyl, urazolyl, azetidinyl, pyrazolidinyl, imidazolidinyl, oxazolidinyl, isoxazolinyl, isoxazolyl, thiazolidinyl, isothiazolyl, isothiazolinyl, oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, or piperidin-2-onyl (valerolactam).

The compound wherein R₂ is NH₂⁺, OH, SH, NOH, NHNH₂, NHNH₃⁺, CO₂H, SO₂OH, B(OH)₂, or imidazolium.

In another example, R₃ can be N, NH, NH₂⁺, O, OH, S, SH, C—R₁₃, CH—R₁₃, N—R₁₃, NH—R₁₃, O—R₁₃, or S—R₁₃, wherein R₁₃ is NH₂⁺, NH₃⁺, CO₂H, B(OH)₂, CH(NH₂)₂, C(NH₂)₂⁺, CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present.

In a further example, R₁₀ can be N, NH, O, or S. In a further example, R₇ can be CH.

It is to be understood that while a particular moiety or group can be referred to herein as a hydrogen bond donor or acceptor, this terminology is used to merely categorize the various substituents for ease of reference. Such language should not be interpreted to mean that a particular moiety actually participates in hydrogen bonding with the riboswitch or some other compound. It is possible that, for example, a moiety referred to herein as a hydrogen bond acceptor (or donor) could solely or additionally be involved in hydrophobic, ionic, van de Waals, or other type of interaction with the riboswitch or other compound.

It is also understood that certain groups disclosed herein can be referred to herein as both a hydrogen bond acceptor and a hydrogen bond donor. For example, —OH can be a hydrogen bond donor by donating the hydrogen atom; —OH can also be a hydrogen bond acceptor through one or more of the nonbonded electron pairs on the oxygen atom. Thus, throughout the specification various moieties can be a hydrogen bond donor and acceptor and can be referred to as such.

Every compound within the above definition is intended to be and should be considered to be specifically disclosed herein. Further, every subgroup that can be identified within the above definition is intended to be and should be considered to be specifically disclosed herein. As a result, it is specifically contemplated that any compound, or subgroup of compounds can be either specifically included for or excluded from use or included in or excluded from a list of compounds. As an example, a group of compounds is contemplated where each compound is as defined above and is able to activate a lysine-responsive riboswitch.

It should be understood that particular contacts and interactions (such as hydrogen bond donation or acceptance) described herein for compounds interacting with riboswitches are preferred but are not essential for interaction of a compound with a riboswitch. For example, compounds can interact with riboswitches with less affinity and/or specificity than compounds having the disclosed contacts and interactions. Further, different or additional functional groups on the compounds can introduce new, different and/or compensating contacts with the riboswitches. For example, for lysine riboswitches, large functional groups can be used. Such functional groups can have, and can be designed to have, contacts and interactions with other part of the riboswitch. Such contacts and interactions can compensate for contacts and interactions of the trigger molecules and core structure.

### D. Constructs, Vectors and Expression Systems

The disclosed lysine riboswitches can be used with any suitable expression system. Recombinant expression is usefully accomplished using a vector, such as a plasmid. The vector can include a promoter operably linked to riboswitch-encoding sequence and RNA to be expression (e.g., RNA encoding a protein). The vector can also include other elements required for transcription and translation. As used herein, vector refers to any carrier containing exogenous DNA. Thus, vectors are agents that transport the exogenous nucleic acid into a cell without degradation and include a promoter yielding expression of the nucleic acid in the cells into which it is delivered. Vectors include but are not limited to plasmids, viral nucleic acids, viruses, phage nucleic acids, phages, cosmids, and artificial chromosomes. A variety of prokaryotic and eukaryotic expression vectors suitable for carrying riboswitch-regulated constructs can be produced. Such expression vectors include, for example, pET, pET3d, pCR2.1, pBAD, pUC, and yeast vectors. The vectors can be used, for example, in a variety of in vivo and in vitro situation.

Viral vectors include adenovirus, adeno-associated virus, herpes virus, vaccinia virus, polio virus, AIDS virus, neuronal trophic virus, Sindbis and other RNA viruses, including these viruses with the HIV backbone. Also useful are any viral families which share the properties of these viruses which make them suitable for use as vectors. Retroviral vectors, which are described in Verma (1985), include Murine Maloney Leukemia virus, MMLV, and retroviruses that express the desirable properties of MMLV as a vector. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA.

A “promoter” is generally a sequence or sequences of DNA that function when in a relatively fixed location in regard to the transcription start site. A “promoter” contains core elements required for basic interaction of RNA polymerase and transcription factors and can contain upstream elements and response elements.

“Enhancer” generally refers to a sequence of DNA that functions at no fixed distance from the transcription start site and can be either 5′ (Laimins, 1981) or 3′ (Lusky et al., 1983) to the transcription unit. Furthermore, enhancers can be within an intron (Banerji et al., 1983) as well as within the coding sequence itself (Osborne et al., 1984). They are usually between 10 and 300 by in length, and they function in cis. Enhancers function to increase transcription from nearby promoters. Enhancers, like promoters, also often contain response elements that mediate the regulation of transcription. Enhancers often determine the regulation of expression.

Expression vectors used in eukaryotic host cells (yeast, fungi, insect, plant, animal, human or nucleated cells) can also contain sequences necessary for the termination of transcription which can affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs.

The vector can include nucleic acid sequence encoding a marker product. This marker product is used to determine if the gene has been delivered to the cell and once delivered is being expressed. Preferred marker genes are the E. Coli lacZ gene which encodes β-galactosidase and green fluorescent protein.

In some embodiments the marker can be a selectable marker. When such selectable markers are successfully transferred into a host cell, the transformed host cell can survive if placed under selective pressure. There are two widely used distinct categories of selective regimes. The first category is based on a cell's metabolism and the use of a mutant cell line which lacks the ability to grow independent of a supplemented media. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern and Berg, 1982), mycophenolic acid, (Mulligan and Berg, 1980) or hygromycin (Sugden et al., 1985).

Gene transfer can be obtained using direct transfer of genetic material, in but not limited to, plasmids, viral vectors, viral nucleic acids, phage nucleic acids, phages, cosmids, and artificial chromosomes, or via transfer of genetic material in cells or carriers such as cationic liposomes. Such methods are well known in the art and readily adaptable for use in the method described herein. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)). Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991).

1. Viral Vectors

Preferred viral vectors are Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Preferred retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors have higher transaction (ability to introduce genes) abilities than do most chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

i. Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

ii. Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A preferred viral vector is one based on an adenovirus which has had the E1 gene removed and these virons are generated in a cell line such as the human 293 cell line. In another preferred embodiment both the E1 and E3 genes are removed from the adenovirus genome.

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and can contain upstream elements and response elements.

2. Viral Promoters and Enhancers

Preferred promoters controlling transcription from vectors in mammalian host cells can be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lucky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 by in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus. Preferred examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer can be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

It is preferred that the promoter and/or enhancer region be active in all eukaryotic cell types. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) can also contain sequences necessary for the termination of transcription which can affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In a preferred embodiment of the transcription unit, the polyadenylation region is derived from the SV40 early polyadenylation signal and consists of about 400 bases. It is also preferred that the transcribed units contain other standard sequences alone or in combination with the above sequences improve expression from, or stability of, the construct.

3. Markers

The vectors can include nucleic acid sequence encoding a marker product. This marker product is used to determine if the gene has been delivered to the cell and once delivered is being expressed. Preferred marker genes are the E. Coli lacZ gene which encodes β-galactosidase and green fluorescent protein.

In some embodiments the marker can be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR⁻ cells and mouse LTK⁻ cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

### E. Biosensor Riboswitches

Also disclosed are biosensor riboswitches. Biosensor riboswitches are engineered riboswitches that produce a detectable signal in the presence of their cognate trigger molecule. Useful biosensor riboswitches can be triggered at or above threshold levels of the trigger molecules. Biosensor riboswitches can be designed for use in vivo or in vitro. For example, lysine biosensor riboswitches operably linked to a reporter RNA that encodes a protein that serves as or is involved in producing a signal can be used in vivo by engineering a cell or organism to harbor a nucleic acid construct encoding the lysine riboswitch/reporter RNA. An example of a biosensor riboswitch for use in vitro is a riboswitch that includes a conformation dependent label, the signal from which changes depending on the activation state of the riboswitch. Such a biosensor riboswitch preferably uses an aptamer domain from or derived from a naturally occurring riboswitch, such as lysine.

### F. Reporter Proteins and Peptides

For assessing activation of a riboswitch, or for biosensor riboswitches, a reporter protein or peptide can be used. The reporter protein or peptide can be encoded by the RNA the expression of which is regulated by the riboswitch. The examples describe the use of some specific reporter proteins. The use of reporter proteins and peptides is well known and can be adapted easily for use with riboswitches. The reporter proteins can be any protein or peptide that can be detected or that produces a detectable signal. Preferably, the presence of the protein or peptide can be detected using standard techniques (e.g., radioimmunoassay, radio-labeling, immunoassay, assay for enzymatic activity, absorbance, fluorescence, luminescence, and Western blot). More preferably, the level of the reporter protein is easily quantifiable using standard techniques even at low levels. Useful reporter proteins include luciferases, green fluorescent proteins and their derivatives, such as firefly luciferase (FL) from Photinus pyralis, and Renilla luciferase (RL) from Renilla reniformis.

### G. Conformation Dependent Labels

Conformation dependent labels refer to all labels that produce a change in fluorescence intensity or wavelength based on a change in the form or conformation of the molecule or compound (such as a riboswitch) with which the label is associated. Examples of conformation dependent labels used in the context of probes and primers include molecular beacons, Amplifluors, FRET probes, cleavable FRET probes, TaqMan probes, scorpion primers, fluorescent triplex oligos including but not limited to triplex molecular beacons or triplex FRET probes, fluorescent water-soluble conjugated polymers, PNA probes and QPNA probes. Such labels, and, in particular, the principles of their function, can be adapted for use with riboswitches. Several types of conformation dependent labels are reviewed in Schweitzer and Kingsmore, Curr. Opin. Biotech. 12:21-27 (2001).

Stem quenched labels, a form of conformation dependent labels, are fluorescent labels positioned on a nucleic acid such that when a stem structure forms a quenching moiety is brought into proximity such that fluorescence from the label is quenched. When the stem is disrupted (such as when a riboswitch containing the label is activated), the quenching moiety is no longer in proximity to the fluorescent label and fluorescence increases. Examples of this effect can be found in molecular beacons, fluorescent triplex oligos, triplex molecular beacons, triplex FRET probes, and QPNA probes, the operational principles of which can be adapted for use with riboswitches.

Stem activated labels, a form of conformation dependent labels, are labels or pairs of labels where fluorescence is increased or altered by formation of a stem structure. Stem activated labels can include an acceptor fluorescent label and a donor moiety such that, when the acceptor and donor are in proximity (when the nucleic acid strands containing the labels form a stem structure), fluorescence resonance energy transfer from the donor to the acceptor causes the acceptor to fluoresce. Stem activated labels are typically pairs of labels positioned on nucleic acid molecules (such as riboswitches) such that the acceptor and donor are brought into proximity when a stem structure is formed in the nucleic acid molecule. If the donor moiety of a stem activated label is itself a fluorescent label, it can release energy as fluorescence (typically at a different wavelength than the fluorescence of the acceptor) when not in proximity to an acceptor (that is, when a stem structure is not formed). When the stem structure forms, the overall effect would then be a reduction of donor fluorescence and an increase in acceptor fluorescence. FRET probes are an example of the use of stem activated labels, the operational principles of which can be adapted for use with riboswitches.

### H. Detection Labels

To aid in detection and quantitation of riboswitch activation, deactivation or blocking, or expression of nucleic acids or protein produced upon activation, deactivation or blocking of riboswitches, detection labels can be incorporated into detection probes or detection molecules or directly incorporated into expressed nucleic acids or proteins. As used herein, a detection label is any molecule that can be associated with nucleic acid or protein, directly or indirectly, and which results in a measurable, detectable signal, either directly or indirectly. Many such labels are known to those of skill in the art. Examples of detection labels suitable for use in the disclosed method are radioactive isotopes, fluorescent molecules, phosphorescent molecules, enzymes, antibodies, and ligands.

Examples of suitable fluorescent labels include fluorescein isothiocyanate (FITC), 5,6-carboxymethyl fluorescein, Texas red, nitrobenz-2-oxa-1,3-diazol-4-yl (NBD), coumarin, dansyl chloride, rhodamine, amino-methyl coumarin (AMCA), Eosin, Erythrosin, BODIPY®, Cascade Blue®, Oregon Green®, pyrene, lissamine, xanthenes, acridines, oxazines, phycoerythrin, macrocyclic chelates of lanthanide ions such as quantum Dye™, fluorescent energy transfer dyes, such as thiazole orange-ethidium heterodimer, and the cyanine dyes Cy3, Cy3.5, Cy5, Cy5.5 and Cy7. Examples of other specific fluorescent labels include 3-Hydroxypyrene 5,8,10-Tri Sulfonic acid, 5-Hydroxy Tryptamine (5-HT), Acid Fuchsin, Alizarin Complexon, Alizarin Red, Allophycocyanin, Aminocoumarin, Anthroyl Stearate, Astrazon Brilliant Red 4G, Astrazon Orange R, Astrazon Red 6B, Astrazon Yellow 7 GLL, Atabrine, Auramine, Aurophosphine, Aurophosphine G, BAO 9 (Bisaminophenyloxadiazole), BCECF, Berberine Sulphate, Bisbenzamide, Blancophor FFG Solution, Blancophor SV, Bodipy F1, Brilliant Sulphoflavin FF, Calcien Blue, Calcium Green, Calcofluor RW Solution, Calcofluor White, Calcophor White ABT Solution, Calcophor White Standard Solution, Carbostyryl, Cascade Yellow, Catecholamine, Chinacrine, Coriphosphine O, Coumarin-Phalloidin, CY3.1 8, CY5.1 8, CY7, Dans (1-Dimethyl Amino Naphaline 5 Sulphonic Acid), Dansa (Diamino Naphtyl Sulphonic Acid), Dansyl NH—CH3, Diamino Phenyl Oxydiazole (DAO), Dimethylamino-5-Sulphonic acid, Dipyrrometheneboron Difluoride, Diphenyl Brilliant Flavine 7GFF, Dopamine, Erythrosin ITC, Euchrysin, FIF (Formaldehyde Induced Fluorescence), Flazo Orange, Fluo 3, Fluorescamine, Fura-2, Genacryl Brilliant Red B, Genacryl Brilliant Yellow 10GF, Genacryl Pink 3G, Genacryl Yellow 5GF, Gloxalic Acid, Granular Blue, Haematoporphyrin, Indo-1, Intrawhite Cf Liquid, Leucophor PAF, Leucophor SF, Leucophor WS, Lissamine Rhodamine B200 (RD200), Lucifer Yellow CH, Lucifer Yellow VS, Magdala Red, Marina Blue, Maxilon Brilliant Flavin 10 GFF, Maxilon Brilliant Flavin 8 GFF, MPS (Methyl Green Pyronine Stilbene), Mithramycin, NBD Amine, Nitrobenzoxadidole, Noradrenaline, Nuclear Fast Red, Nuclear Yellow, Nylosan Brilliant Flavin E8G, Oxadiazole, Pacific Blue, Pararosaniline (Feulgen), Phorwite AR Solution, Phorwite BKL, Phorwite Rev, Phorwite RPA, Phosphine 3R, Phthalocyanine, Phycoerythrin R, Polyazaindacene Pontochrome Blue Black, Porphyrin, Primuline, Procion Yellow, Pyronine, Pyronine B, Pyrozal Brilliant Flavin 7GF, Quinacrine Mustard, Rhodamine 123, Rhodamine 5 GLD, Rhodamine 6G, Rhodamine B, Rhodamine B 200, Rhodamine B Extra, Rhodamine BB, Rhodamine BG, Rhodamine WT, Serotonin, Sevron Brilliant Red 2B, Sevron Brilliant Red 4G, Sevron Brilliant Red B, Sevron Orange, Sevron Yellow L, SITS (Primuline), SITS (Stilbene Isothiosulphonic acid), Stilbene, Snarf 1, sulpho Rhodamine B Can C, Sulpho Rhodamine G Extra, Tetracycline, Thiazine Red R, Thioflavin S, Thioflavin TCN, Thioflavin 5, Thiolyte, Thiozol Orange, Tinopol CBS, True Blue, Ultralite, Uranine B, Uvitex SFC, Xylene Orange, and XRITC.

Useful fluorescent labels are fluorescein (5-carboxyfluorescein-N-hydroxysuccinimide ester), rhodamine (5,6-tetramethyl rhodamine), and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. Other examples of fluorescein dyes include 6-carboxyfluorescein (6-FAM), 2′,4′,1,4,-tetrachlorofluorescein (TET), 2′,4′,5′,7′,1,4-hexachlorofluorescein (HEX), 2′,7′-dimethoxy-4′,5′-dichloro-6-carboxyrhodamine (JOE), 2′-chloro-5′-fluoro-7′,8′-fused phenyl-1,4-dichloro-6-carboxyfluorescein (NED), and 2′-chloro-7′-phenyl-1,4-dichloro-6-carboxyfluorescein (VIC). Fluorescent labels can be obtained from a variety of commercial sources, including Amersham Pharmacia Biotech, Piscataway, N.J.; Molecular Probes, Eugene, Oreg.; and Research Organics, Cleveland, Ohio.

Additional labels of interest include those that provide for signal only when the probe with which they are associated is specifically bound to a target molecule, where such labels include: “molecular beacons” as described in Tyagi & Kramer, Nature Biotechnology (1996) 14:303 and EP 0 070 685 B1. Other labels of interest include those described in U.S. Pat. No. 5,563,037; WO 97/17471 and WO 97/17076.

Labeled nucleotides are a useful form of detection label for direct incorporation into expressed nucleic acids during synthesis. Examples of detection labels that can be incorporated into nucleic acids include nucleotide analogs such as BrdUrd (5-bromodeoxyuridine, Hoy and Schimke, Mutation Research 290:217-230 (1993)), aminoallyldeoxyuridine (Henegariu et al., Nature Biotechnology 18:345-348 (2000)), 5-methylcytosine (Sano et al., Biochim. Biophys. Acta 951:157-165 (1988)), bromouridine (Wansick et al., J. Cell Biology 122:283-293 (1993)) and nucleotides modified with biotin (Langer et al., Proc. Natl. Acad. Sci. USA 78:6633 (1981)) or with suitable haptens such as digoxygenin (Kerkhof, Anal. Biochem. 205:359-364 (1992)). Suitable fluorescence-labeled nucleotides are Fluorescein-isothiocyanate-dUTP, Cyanine-3-dUTP and Cyanine-5-dUTP (Yu et al., Nucleic Acids Res., 22:3226-3232 (1994)). A preferred nucleotide analog detection label for DNA is BrdUrd (bromodeoxyuridine, BrdUrd, BrdU, BUdR, Sigma-Aldrich Co). Other useful nucleotide analogs for incorporation of detection label into DNA are AA-dUTP (aminoallyl-deoxyuridine triphosphate, Sigma-Aldrich Co.), and 5-methyl-dCTP (Roche Molecular Biochemicals). A useful nucleotide analog for incorporation of detection label into RNA is biotin-16-UTP (biotin-16-uridine-5′-triphosphate, Roche Molecular Biochemicals). Fluorescein, Cy3, and Cy5 can be linked to dUTP for direct labeling. Cy3.5 and Cy7 are available as avidin or anti-digoxygenin conjugates for secondary detection of biotin- or digoxygenin-labeled probes.

Detection labels that are incorporated into nucleic acid, such as biotin, can be subsequently detected using sensitive methods well-known in the art. For example, biotin can be detected using streptavidin-alkaline phosphatase conjugate (Tropix, Inc.), which is bound to the biotin and subsequently detected by chemiluminescence of suitable substrates (for example, chemiluminescent substrate CSPD: disodium, 3-(4-methoxyspiro-[1,2,-dioxetane-3-2′-(5′-chloro)tricyclo [3.3.1.1^{3,7}]decane]-4-yl)phenyl phosphate; Tropix, Inc.). Labels can also be enzymes, such as alkaline phosphatase, soybean peroxidase, horseradish peroxidase and polymerases, that can be detected, for example, with chemical signal amplification or by using a substrate to the enzyme which produces light (for example, a chemiluminescent 1,2-dioxetane substrate) or fluorescent signal.

Molecules that combine two or more of these detection labels are also considered detection labels. Any of the known detection labels can be used with the disclosed probes, tags, molecules and methods to label and detect activated or deactivated riboswitches or nucleic acid or protein produced in the disclosed methods. Methods for detecting and measuring signals generated by detection labels are also known to those of skill in the art. For example, radioactive isotopes can be detected by scintillation counting or direct visualization; fluorescent molecules can be detected with fluorescent spectrophotometers; phosphorescent molecules can be detected with a spectrophotometer or directly visualized with a camera; enzymes can be detected by detection or visualization of the product of a reaction catalyzed by the enzyme; antibodies can be detected by detecting a secondary detection label coupled to the antibody. As used herein, detection molecules are molecules which interact with a compound or composition to be detected and to which one or more detection labels are coupled.

### I. Sequence Similarities

It is understood that as discussed herein the use of the terms homology and identity mean the same thing as similarity. Thus, for example, if the use of the word homology is used between two sequences (non-natural sequences, for example) it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed riboswitches, aptamers, expression platforms, genes and proteins herein, is through defining the variants and derivatives in terms of homology to specific known sequences. This identity of particular sequences disclosed herein is also discussed elsewhere herein. In general, variants of riboswitches, aptamers, expression platforms, genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to a stated sequence or a native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison can be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods can differ, but the skilled artisan understands if identity is found with at least one of these methods, the sequences would be said to have the stated identity.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

### J. Hybridization and Selective Hybridization

The term hybridization typically means a sequence driven interaction between at least two nucleic acid molecules, such as a primer or a probe and a riboswitch or a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization can involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting nucleic acid is in for example, 10 or 100 or 1000 fold excess. This type of assay can be performed at under conditions where both the limiting and non-limiting nucleic acids are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of nucleic acid that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the nucleic acid is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the nucleic acid molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions can provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

### K. Nucleic Acids

There are a variety of molecules disclosed herein that are nucleic acid based, including, for example, riboswitches, aptamers, and nucleic acids that encode riboswitches and aptamers. The disclosed nucleic acids can be made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if a nucleic acid molecule is introduced into a cell or cell environment through for example exogenous delivery, it is advantageous that the nucleic acid molecule be made up of nucleotide analogs that reduce the degradation of the nucleic acid molecule in the cellular environment.

So long as their relevant function is maintained, riboswitches, aptamers, expression platforms and any other oligonucleotides and nucleic acids can be made up of or include modified nucleotides (nucleotide analogs). Many modified nucleotides are known and can be used in oligonucleotides and nucleic acids. A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl, hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine. 5-methylcytosine can increase the stability of duplex formation. Other modified bases are those that function as universal bases. Universal bases include 3-nitropyrrole and 5-nitroindole. Universal bases substitute for the normal bases but have no bias in base pairing. That is, universal bases can base pair with any other base. Base modifications often can be combined with for example a sugar modification, such as 2′-O-methoxyethyl, to achieve unique properties such as increased duplex stability. There are numerous United States patents such as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, which detail and describe a range of base modifications. Each of these patents is herein incorporated by reference in its entirety, and specifically for their description of base modifications, their synthesis, their use, and their incorporation into oligonucleotides and nucleic acids.

Nucleotide analogs can also include modifications of the sugar moiety. Modifications to the sugar moiety would include natural modifications of the ribose and deoxyribose as well as synthetic modifications. Sugar modifications include but are not limited to the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C1 to C10, alkyl or C2 to C10 alkenyl and alkynyl. 2′ sugar modifications also include but are not limited to —O[(CH₂)nO]mCH₃, —O(CH₂)nOCH₃, —O(CH₂)nNH₂, —O(CH₂)nCH₃, —O(CH₂)n—ONH₂, and —O(CH₂)nON[(CH₂)nCH₃)]₂, where n and m are from 1 to about 10.

Other modifications at the 2′ position include but are not limited to: C1 to C10 lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications can also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH₂ and S, Nucleotide sugar analogs can also have sugar mimetics such as cyclobutyl moieties in place of the pentofuranosyl sugar. There are numerous United States patents that teach the preparation of such modified sugar structures such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety, and specifically for their description of modified sugar structures, their synthesis, their use, and their incorporation into nucleotides, oligonucleotides and nucleic acids.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkages between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference its entirety, and specifically for their description of modified phosphates, their synthesis, their use, and their incorporation into nucleotides, oligonucleotides and nucleic acids.

It is understood that nucleotide analogs need only contain a single modification, but can also contain multiple modifications within one of the moieties or between different moieties.

Nucleotide substitutes are molecules having similar functional properties to nucleotides, but which do not contain a phosphate moiety, such as peptide nucleic acid (PNA). Nucleotide substitutes are molecules that will recognize and hybridize to (base pair to) complementary nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference its entirety, and specifically for their description of phosphate replacements, their synthesis, their use, and their incorporation into nucleotides, oligonucleotides and nucleic acids.

It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. (See also Nielsen et al., Science 254:1497-1500 (1991)).

Oligonucleotides and nucleic acids can be comprised of nucleotides and can be made up of different types of nucleotides or the same type of nucleotides. For example, one or more of the nucleotides in an oligonucleotide can be ribonucleotides, 2′-O-methyl ribonucleotides, or a mixture of ribonucleotides and 2′-O-methyl ribonucleotides; about 10% to about 50% of the nucleotides can be ribonucleotides, 2′-O-methyl ribonucleotides, or a mixture of ribonucleotides and 2′-O-methyl ribonucleotides; about 50% or more of the nucleotides can be ribonucleotides, 2′-O-methyl ribonucleotides, or a mixture of ribonucleotides and 2′-O-methyl ribonucleotides; or all of the nucleotides are ribonucleotides, 2′-O-methyl ribonucleotides, or a mixture of ribonucleotides and 2′-β-methyl ribonucleotides. Such oligonucleotides and nucleic acids can be referred to as chimeric oligonucleotides and chimeric nucleic acids.

### L. Solid Supports

Solid supports are solid-state substrates or supports with which molecules (such as trigger molecules) and riboswitches (or other components used in, or produced by, the disclosed methods) can be associated. Riboswitches and other molecules can be associated with solid supports directly or indirectly. For example, analytes (e.g., trigger molecules, test compounds) can be bound to the surface of a solid support or associated with capture agents (e.g., compounds or molecules that bind an analyte) immobilized on solid supports. As another example, riboswitches can be bound to the surface of a solid support or associated with probes immobilized on solid supports. An array is a solid support to which multiple riboswitches, probes or other molecules have been associated in an array, grid, or other organized pattern.

Solid-state substrates for use in solid supports can include any solid material with which components can be associated, directly or indirectly. This includes materials such as acrylamide, agarose, cellulose, nitrocellulose, glass, gold, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, functionalized silane, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or a combination. Solid-state substrates and solid supports can be porous or non-porous. A chip is a rectangular or square small piece of material. Preferred forms for solid-state substrates are thin films, beads, or chips. A useful form for a solid-state substrate is a microtiter dish. In some embodiments, a multiwell glass slide can be employed.

An array can include a plurality of riboswitches, trigger molecules, other molecules, compounds or probes immobilized at identified or predefined locations on the solid support. Each predefined location on the solid support generally has one type of component (that is, all the components at that location are the same). Alternatively, multiple types of components can be immobilized in the same predefined location on a solid support. Each location will have multiple copies of the given components. The spatial separation of different components on the solid support allows separate detection and identification.

Although useful, it is not required that the solid support be a single unit or structure. A set of riboswitches, trigger molecules, other molecules, compounds and/or probes can be distributed over any number of solid supports. For example, at one extreme, each component can be immobilized in a separate reaction tube or container, or on separate beads or microparticles.

Methods for immobilization of oligonucleotides to solid-state substrates are well established. Oligonucleotides, including address probes and detection probes, can be coupled to substrates using established coupling methods. For example, suitable attachment methods are described by Pease et al., Proc. Natl. Acad. Sci. USA 91(10:5022-5026 (1994), and Khrapko et al., Mol Biol (Mosk) (USSR) 25:718-730 (1991). A method for immobilization of 3′-amine oligonucleotides on casein-coated slides is described by Stimpson et al., Proc. Natl. Acad. Sci. USA 92:6379-6383 (1995). A useful method of attaching oligonucleotides to solid-state substrates is described by Guo et al., Nucleic Acids Res. 22:5456-5465 (1994).

Each of the components (for example, riboswitches, trigger molecules, or other molecules) immobilized on the solid support can be located in a different predefined region of the solid support. The different locations can be different reaction chambers. Each of the different predefined regions can be physically separated from each other of the different regions. The distance between the different predefined regions of the solid support can be either fixed or variable. For example, in an array, each of the components can be arranged at fixed distances from each other, while components associated with beads will not be in a fixed spatial relationship. In particular, the use of multiple solid support units (for example, multiple beads) will result in variable distances.

Components can be associated or immobilized on a solid support at any density. Components can be immobilized to the solid support at a density exceeding 400 different components per cubic centimeter. Arrays of components can have any number of components. For example, an array can have at least 1,000 different components immobilized on the solid support, at least 10,000 different components immobilized on the solid support, at least 100,000 different components immobilized on the solid support, or at least 1,000,000 different components immobilized on the solid support.

### M. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for detecting compounds, the kit comprising one or more biosensor riboswitches. The kits also can contain reagents and labels for detecting activation of the riboswitches.

### N. Mixtures

Disclosed are mixtures formed by performing or preparing to perform the disclosed method. For example, disclosed are mixtures comprising riboswitches and trigger molecules.

Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

### O. Systems

Disclosed are systems useful for performing, or aiding in the performance of, the disclosed method. Systems generally comprise combinations of articles of manufacture such as structures, machines, devices, and the like, and compositions, compounds, materials, and the like. Such combinations that are disclosed or that are apparent from the disclosure are contemplated. For example, disclosed and contemplated are systems comprising biosensor riboswitches, a solid support and a signal-reading device.

### P. Data Structures and Computer Control

Disclosed are data structures used in, generated by, or generated from, the disclosed method. Data structures generally are any form of data, information, and/or objects collected, organized, stored, and/or embodied in a composition or medium. Riboswitch structures and activation measurements stored in electronic form, such as in RAM or on a storage disk, is a type of data structure.

The disclosed method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

### Methods

Disclosed are methods for activating, deactivating or blocking a riboswitch. Such methods can involve, for example, bringing into contact a riboswitch and a compound or trigger molecule that can activate, deactivate or block the riboswitch. Riboswitches function to control gene expression through the binding or removal of a trigger molecule. Compounds can be used to activate, deactivate or block a riboswitch. The trigger molecule for a riboswitch (as well as other activating compounds) can be used to activate a riboswitch. Compounds other than the trigger molecule generally can be used to deactivate or block a riboswitch. Riboswitches can also be deactivated by, for example, removing trigger molecules from the presence of the riboswitch. Thus, the disclosed method of deactivating a riboswitch can involve, for example, removing a trigger molecule (or other activating compound) from the presence or contact with the riboswitch. A riboswitch can be blocked by, for example, binding of an analog of the trigger molecule that does not activate the riboswitch.

Also disclosed are methods for altering expression of an RNA molecule, or of a gene encoding an RNA molecule, where the RNA molecule includes a riboswitch, by bringing a compound into contact with the RNA molecule. Riboswitches function to control gene expression through the binding or removal of a trigger molecule. Thus, subjecting an RNA molecule of interest that includes a riboswitch to conditions that activate, deactivate or block the riboswitch can be used to alter expression of the RNA. Expression can be altered as a result of, for example, termination of transcription or blocking of ribosome binding to the RNA. Binding of a trigger molecule can, depending on the nature of the riboswitch, reduce or prevent expression of the RNA molecule or promote or increase expression of the RNA molecule.

Also disclosed are methods for regulating expression of a naturally occurring gene or RNA that contains a riboswitch by activating, deactivating or blocking the riboswitch. If the gene is essential for survival of a cell or organism that harbors it, activating, deactivating or blocking the riboswitch can result in death, stasis or debilitation of the cell or organism. For example, activating a naturally occurring riboswitch in a naturally occurring gene that is essential to survival of a microorganism can result in death of the microorganism (if activation of the riboswitch turns off or represses expression). This is one basis for the use of the disclosed compounds and methods for antimicrobial and antibiotic effects. The compounds that have these antimicrobial effects are considered to be bacteriostatic or bacteriocidal.

Also disclosed are methods for selecting and identifying compounds that can activate, deactivate or block a riboswitch. Activation of a riboswitch refers to the change in state of the riboswitch upon binding of a trigger molecule. A riboswitch can be activated by compounds other than the trigger molecule and in ways other than binding of a trigger molecule. The term trigger molecule is used herein to refer to molecules and compounds that can activate a riboswitch. This includes the natural or normal trigger molecule for the riboswitch and other compounds that can activate the riboswitch. Natural or normal trigger molecules are the trigger molecule for a given riboswitch in nature or, in the case of some non-natural riboswitches, the trigger molecule for which the riboswitch was designed or with which the riboswitch was selected (as in, for example, in vitro selection or in vitro evolution techniques). Non-natural trigger molecules can be referred to as non-natural trigger molecules.

Also disclosed are methods of killing or inhibiting bacteria, comprising contacting the bacteria with a compound disclosed herein or identified by the methods disclosed herein.

Also disclosed are methods of identifying compounds that activate, deactivate or block a riboswitch. For example, compounds that activate a riboswitch can be identified by bringing into contact a test compound and a riboswitch and assessing activation of the riboswitch. If the riboswitch is activated, the test compound is identified as a compound that activates the riboswitch. Activation of a riboswitch can be assessed in any suitable manner. For example, the riboswitch can be linked to a reporter RNA and expression, expression level, or change in expression level of the reporter RNA can be measured in the presence and absence of the test compound. As another example, the riboswitch can include a conformation dependent label, the signal from which changes depending on the activation state of the riboswitch. Such a riboswitch preferably uses an aptamer domain from or derived from a naturally occurring riboswitch. As can be seen, assessment of activation of a riboswitch can be performed with the use of a control assay or measurement or without the use of a control assay or measurement. Methods for identifying compounds that deactivate a riboswitch can be performed in analogous ways.

In addition to the methods disclosed elsewhere herein, identification of compounds that block a riboswitch can be accomplished in any suitable manner. For example, an assay can be performed for assessing activation or deactivation of a riboswitch in the presence of a compound known to activate or deactivate the riboswitch and in the presence of a test compound. If activation or deactivation is not observed as would be observed in the absence of the test compound, then the test compound is identified as a compound that blocks activation or deactivation of the riboswitch.

Also disclosed are methods of detecting compounds using biosensor riboswitches. The method can include bringing into contact a test sample and a biosensor riboswitch and assessing the activation of the biosensor riboswitch. Activation of the biosensor riboswitch indicates the presence of the trigger molecule for the biosensor riboswitch in the test sample. Biosensor riboswitches are engineered riboswitches that produce a detectable signal in the presence of their cognate trigger molecule. Useful biosensor riboswitches can be triggered at or above threshold levels of the trigger molecules. Biosensor riboswitches can be designed for use in vivo or in vitro. For example, lysine biosensor riboswitches operably linked to a reporter RNA that encodes a protein that serves as or is involved in producing a signal can be used in vivo by engineering a cell or organism to harbor a nucleic acid construct encoding the riboswitch/reporter RNA. An example of a biosensor riboswitch for use in vitro is a lysine riboswitch that includes a conformation dependent label, the signal from which changes depending on the activation state of the riboswitch. Such a biosensor riboswitch preferably uses an aptamer domain from or derived from a naturally occurring lysine riboswitch.

Also disclosed are compounds made by identifying a compound that activates, deactivates or blocks a riboswitch and manufacturing the identified compound. This can be accomplished by, for example, combining compound identification methods as disclosed elsewhere herein with methods for manufacturing the identified compounds. For example, compounds can be made by bringing into contact a test compound and a riboswitch, assessing activation of the riboswitch, and, if the riboswitch is activated by the test compound, manufacturing the test compound that activates the riboswitch as the compound.

Also disclosed are compounds made by checking activation, deactivation or blocking of a riboswitch by a compound and manufacturing the checked compound. This can be accomplished by, for example, combining compound activation, deactivation or blocking assessment methods as disclosed elsewhere herein with methods for manufacturing the checked compounds. For example, compounds can be made by bringing into contact a test compound and a riboswitch, assessing activation of the riboswitch, and, if the riboswitch is activated by the test compound, manufacturing the test compound that activates the riboswitch as the compound. Checking compounds for their ability to activate, deactivate or block a riboswitch refers to both identification of compounds previously unknown to activate, deactivate or block a riboswitch and to assessing the ability of a compound to activate, deactivate or block a riboswitch where the compound was already known to activate, deactivate or block the riboswitch.

Disclosed is a method of detecting a compound of interest, the method comprising bringing into contact a sample and a lysine riboswitch, wherein the riboswitch is activated by the compound of interest, wherein the riboswitch produces a signal when activated by the compound of interest, wherein the riboswitch produces a signal when the sample contains the compound of interest. The riboswitch can change conformation when activated by the compound of interest, wherein the change in conformation produces a signal via a conformation dependent label. The riboswitch can change conformation when activated by the compound of interest, wherein the change in conformation causes a change in expression of an RNA linked to the riboswitch, wherein the change in expression produces a signal. The signal can be produced by a reporter protein expressed from the RNA linked to the riboswitch.

Disclosed is a method comprising (a) testing a compound for inhibition of gene expression of a gene encoding an RNA comprising a riboswitch, wherein the inhibition is via the riboswitch, and (b) inhibiting gene expression by bringing into contact a cell and a compound that inhibited gene expression in step (a), wherein the cell comprises a gene encoding an RNA comprising a riboswitch, wherein the compound inhibits expression of the gene by binding to the riboswitch.

### A. Identification of Antimicrobial Compounds

Riboswitches are a new class of structured RNAs that have evolved for the purpose of binding small organic molecules. The natural binding pocket of riboswitches can be targeted with metabolite analogs or by compounds that mimic the shape-space of the natural metabolite. The small molecule ligands of riboswitches provide useful sites for derivitization to produce drug candidates. Distribution of some riboswitches is shown in Table 1 of U.S. Application Publication No. 2005-0053951. Once a class of riboswitch has been identified and its potential as a drug target assessed, such as the lysine riboswitch, candidate molecules can be identified.

The emergence of drug-resistant stains of bacteria highlights the need for the identification of new classes of antibiotics. Anti-riboswitch drugs represent a mode of anti-bacterial action that is of considerable interest for the following reasons. Riboswitches control the expression of genes that are critical for fundamental metabolic processes. Therefore manipulation of these gene control elements with drugs yields new antibiotics. These antimicrobial agents can be considered to be bacteriostatic, or bacteriocidal. Riboswitches also carry RNA structures that have evolved to selectively bind metabolites, and therefore these RNA receptors make good drug targets as do protein enzymes and receptors. Furthermore, it has been shown that two antimicrobial compounds (discussed above) kill bacteria by deactivating the antibiotics resistance to emerge through mutation of the RNA target.

A compound can be identified as activating a riboswitch or can be determined to have riboswitch activating activity if the signal in a riboswitch assay is increased in the presence of the compound by at least 1 fold, 2 fold, 3 fold, 4 fold, 5 fold, 50%, 75%, 100%, 125%, 150%, 175%, 200%, 250%, 300%, 400%, or 500% compared to the same riboswitch assay in the absence of the compound (that is, compared to a control assay). The riboswitch assay can be performed using any suitable riboswitch construct. Riboswitch constructs that are particularly useful for riboswitch activation assays are described elsewhere herein. The identification of a compound as activating a riboswitch or as having a riboswitch activation activity can be made in terms of one or more particular riboswitches, riboswitch constructs or classes of riboswitches. For convenience, compounds identified as activating a lysine riboswitch or having riboswitch activating activity for a lysine riboswitch can be so identified for particular lysine riboswitches, such as the lysine riboswitches found in Bacillus anthracis or B. subtilis.

### B. Methods of Using Antimicrobial Compounds

Disclosed herein are in vivo and in vitro anti-bacterial methods. By “anti-bacterial” is meant inhibiting or preventing bacterial growth, killing bacteria, or reducing the number of bacteria. Thus, disclosed is a method of inhibiting or preventing bacterial growth comprising contacting a bacterium with an effective amount of one or more compounds disclosed herein. Additional structures for the disclosed compounds are provided herein.

Disclosed herein is also a method of inhibiting growth of a cell, such as a bacterial cell, that is in a subject, the method comprising administering an effective amount of a compound as disclosed herein to the subject. This can result in the compound being brought into contact with the cell. The subject can have, for example, a bacterial infection, and the bacterial cells can be inhibited by the compound. The bacteria can be any bacteria, such as bacteria from the genus Bacillus, Acinetobacter, Actinobacillus, Clostridium, Desulfitobacterium, Enterococcus, Erwinia, Escherichia, Exiguobacterium, Fusobacterium, Geobacillus, Haemophilus, Klebsiella, Idiomarina, Lactobacillus, Lactococcus, Leuconostoc, Listeria, Moorella, Mycobacterium, Oceanobacillus, Oenococcus, Pasteurella, Pediococcus, Pseudomonas, Shewanella, Shigella, Solibacter, Staphylococcus, Streptococcus, Thermoanaerobacter, Thermotoga, and Vibrio, for example. The bacteria can be, for example, Actinobacillus pleuropneumoniae, Bacillus anthracis, Bacillus cereus, Bacillus clausii, Bacillus halodurans, Bacillus licheniformis, Bacillus subtilis, Bacillus thuringiensis, Clostridium acetobutylicum, Clostridium difficile, Clostridium perfringens, Clostridium tetani, Clostridium thermocellum, Desulfitobacterium hafniense, Enterococcus faecalis, Erwinia carotovora, Escherichia coli, Exiguobacterium sp., Fusobacterium nucleatum, Geobacillus kaustophilus, Haemophilus ducreyi, Haemophilus influenzae, Haemophilus somnus, Idiomarina loihiensis, Lactobacillus acidophilus, Lactobacillus casei, Lactobacillus delbrueckii, Lactobacillus gasseri, Lactobacillus johnsonii, Lactobacillus plantarum, Lactococcus lactis, Leuconostoc mesenteroides, Listeria innocua, Listeria monocytogenes, Moorella thermoacetica, Oceanobacillus iheyensis, Oenococcus oeni, Pasteurella multocida, Pediococcus pentosaceus, Shewanella oneidensis, Shigella flexneri, Solibacter usitatus, Staphylococcus aureus, Staphylococcus epidermidis, Thermoanaerobacter tengcongensis, Thermotoga maritima, Vibrio cholerae, Vibrio fischeri, Vibrio parahaemolyticus, or Vibrio vulnificus. Bacterial growth can also be inhibited in any context in which bacteria are found. For example, bacterial growth in fluids, biofilms, and on surfaces can be inhibited. The compounds disclosed herein can be administered or used in combination with any other compound or composition. For example, the disclosed compounds can be administered or used in combination with another antimicrobial compound.

“Inhibiting bacterial growth” is defined as reducing the ability of a single bacterium to divide into daughter cells, or reducing the ability of a population of bacteria to form daughter cells. The ability of the bacteria to reproduce can be reduced by about 10%, about 20%, about 30%, about 40%, about 50%, about 60%, about 70%, about 80%, about 90%, or 100% or more.

Also provided is a method of inhibiting the growth of and/or killing a bacterium or population of bacteria comprising contacting the bacterium with one or more of the compounds disclosed and described herein.

“Killing a bacterium” is defined as causing the death of a single bacterium, or reducing the number of a plurality of bacteria, such as those in a colony. When the bacteria are referred to in the plural form, the “killing of bacteria” is defined as cell death of a given population of bacteria at the rate of 10% of the population, 20% of the population, 30% of the population, 40% of the population, 50% of the population, 60% of the population, 70% of the population, 80% of the population, 90% of the population, or less than or equal to 100% of the population.

The compounds and compositions disclosed herein have anti-bacterial activity in vitro or in vivo, and can be used in conjunction with other compounds or compositions, which can be bactericidal as well.

By the term “therapeutically effective amount” of a compound as provided herein is meant a nontoxic but sufficient amount of the compound to provide the desired reduction in one or more symptoms. As will be pointed out below, the exact amount of the compound required will vary from subject to subject, depending on the species, age, and general condition of the subject, the severity of the disease that is being treated, the particular compound used, its mode of administration, and the like. Thus, it is not possible to specify an exact “effective amount.” However, an appropriate effective amount may be determined by one of ordinary skill in the art using only routine experimentation.

The compositions and compounds disclosed herein can be administered in vivo in a pharmaceutically acceptable carrier. By “pharmaceutically acceptable” is meant a material that is not biologically or otherwise undesirable, i.e., the material may be administered to a subject without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions or compounds disclosed herein can be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition or compounds, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The compositions and compounds disclosed herein can be used therapeutically in combination with a pharmaceutically acceptable carrier. Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Therapeutic compositions as disclosed herein may also be delivered by the use of monoclonal antibodies as individual carriers to which the compound molecules are coupled. The therapeutic compositions of the present disclosure may also be coupled with soluble polymers as targetable drug carriers. Such polymers can include, but are not limited to, polyvinyl-pyrrolidone, pyran copolymer, polyhydroxypropylmethacryl-amidephenol, polyhydroxyethylaspartamidephenol, or polyethyl-eneoxidepolylysine substituted with palmitoyl residues. Furthermore, the therapeutic compositions of the present disclosure may be coupled to a class of biodegradable polymers useful in achieving controlled release of a drug, for example, polylactic acid, polyepsilon caprolactone, polyhydroxy butyric acid, polyorthoesters, polyacetals, polydihydro-pyrans, polycyanoacrylates and cross-linked or amphipathic block copolymers of hydrogels.

Preferably at least about 3%, more preferably about 10%, more preferably about 20%, more preferably about 30%, more preferably about 50%, more preferably 75% and even more preferably about 100% of the bacterial infection is reduced due to the administration of the compound. A reduction in the infection is determined by such parameters as reduced white blood cell count, reduced fever, reduced inflammation, reduced number of bacteria, or reduction in other indicators of bacterial infection. To increase the percentage of bacterial infection reduction, the dosage can increase to the most effective level that remains non-toxic to the subject.

As used throughout, “subject” refers to an individual. Preferably, the subject is a mammal such as a non-human mammal or a primate, and, more preferably, a human. “Subjects” can include domesticated animals (such as cats, dogs, etc.), livestock (e.g., cattle, horses, pigs, sheep, goats, etc.), laboratory animals (e.g., mouse, rabbit, rat, guinea pig, etc.) and fish.

A “bacterial infection” is defined as the presence of bacteria in a subject or sample. Such bacteria can be an outgrowth of naturally occurring bacteria in or on the subject or sample, or can be due to the invasion of a foreign organism.

The compounds disclosed herein can be used in the same manner as antibiotics. Uses of antibiotics are well established in the art. One example of their use includes treatment of animals. When needed, the disclosed compounds can be administered to the animal via injection or through feed or water, usually with the professional guidance of a veterinarian or nutritionist. They are delivered to animals either individually or in groups, depending on the circumstances such as disease severity and animal species. Treatment and care of the entire herd or flock may be necessary if all animals are of similar immune status and all are exposed to the same disease-causing microorganism.

Another example of a use for the compounds includes reducing a microbial infection of an aquatic animal, comprising the steps of selecting an aquatic animal having a microbial infection, providing an antimicrobial solution comprising a compound as disclosed, chelating agents such as EDTA, TRIENE, adding a pH buffering agent to the solution and adjusting the pH thereof to a value of between about 7.0 and about 9.0, immersing the aquatic animal in the solution and leaving the aquatic animal therein for a period that is effective to reduce the microbial burden of the animal, removing the aquatic animal from the solution and returning the animal to water not containing the solution. The immersion of the aquatic animal in the solution containing the EDTA, a compound as disclosed, and TRIENE and pH buffering agent may be repeated until the microbial burden of the animal is eliminated. (U.S. Pat. No. 6,518,252).

Other uses of the compounds disclosed herein include, but are not limited to, dental treatments and purification of water (this can include municipal water, sewage treatment systems, potable and non-potable water supplies, and hatcheries, for example).

### Specific Embodiments

Disclosed herein is a method of inhibiting gene expression, the method comprising (a) bringing into contact a compound and a cell, (b) wherein the compound has the structure of Formula I:

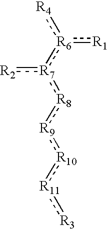

wherein R₂ and R₃ are each independently positively charged, can serve as a hydrogen bond donor, or both,

wherein R₁ is negatively charged, R₄ is negatively charged, or R₁ and R₄ are in a resonance hybrid with a net negative charge,

wherein at least one of R₁ or R₄ can be CH₂, CH₃, NH, O, O⁻, OH, S, S⁻, SH, C—R₁₄, CH—R₁₄, or N—R₁₄, wherein R₁₄ can be CH₂, CH₃, O, O⁻, OH, S, S⁻, or SH,

wherein R₉ can be C, CH, CH₂, NH, O, S, C—R₅, CH—R₅, or N—R₅, wherein R₅ can be methyl, ethyl, propyl, isopropyl, cyclopropyl, butyl, tert-butyl, sec-butyl, iso-butyl, cyclobutyl, ethenyl, 3-propenyl, 1-propenyl, isopropenyl, 3-butenyl, 4-butenyl, 3-propynyl, 3-butynyl, 4-butynyl, diazirinyl, aziridinyl, urazolyl, azetidinyl, pyrazolidinyl, imidazolidinyl, oxazolidinyl, isoxazolinyl, isoxazolyl, thiazolidinyl, isothiazolyl, isothiazolinyl, oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, or piperidin-2-onyl (valerolactam),

wherein R₂ is NH₂⁺, NH₃⁺, O, OH, SH, NOH, NHNH₂, NHNH₃⁺, CO₂H, SO₂OH, B(OH)₂, or imidazolium,

wherein R₃ can be N, NH, NH₂⁺, NH₃⁺, O, OH, S, SH, C—R₁₃, CH—R₁₃, N—R₁₃, NH—O—R₁₃, or S—R₁₃, wherein R₁₃ is NH₂⁺, NH₃⁺, CO₂H, B(OH)₂, CH(NH₂)₂, C(NH₂)₂+₅ CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present,

wherein R₇ can have an S configuration based on a priority of R₂>R₆>R₈,

wherein R₆, R₇, R₈, R₁₀, and R₁₁ each independently can be C, CH, CH₂, N, NH, O, or S,

wherein [symbol] can each independently represent a single or double bond, and

wherein the compound is not lysine, and wherein the cell comprises a gene encoding an RNA comprising a lysine-responsive riboswitch, wherein the compound inhibits expression of the gene by binding to the lysine-responsive riboswitch.

R₃ can be positively charged and can serve as a hydrogen bond donor. R₅ can be uncharged. R₉ can be C, O, or S. The pKₐ of R₃ can be 7 or higher. R₁₃ can be positively charged, and can serve as a hydrogen bond donor, or both.

In one example, R₆, R₇, R₈, R₉, R₁₀ and R₁₁ are not all simultaneously C, CH, or CH₂.

In another example, R₁, R₂, R₃, R₄ and R₉ are not simultaneously O, NH₃⁺, NH₃⁺, O and S, respectively. Furthermore, in another example, R₁, R₂, R₃, and R₄ are not simultaneously O, H, NH₃⁺, and O, respectively. In another example, R₁, R₂, R₃, R₄ and R₉ are not simultaneously CO₂⁻, NH₃⁺, NH₃⁺, and H, respectively. In a further example, R₁, R₂, R₃, R₄ and R₁₁ are not simultaneously O, NH₃⁺, NH₃⁺, O and C—CO₂⁻, respectively. In a further example, R₁, R₂, R₃, and R₄ are not simultaneously NHOH, NH₃⁺, NH₃⁺, O and S, respectively.

In one example, R₉ can be NH, O, S, C—R₅, CH—R₅, or N—R₅, wherein R₅ is methyl, ethyl, propyl, isopropyl, cyclopropyl, butyl, tert-butyl, sec-butyl, iso-butyl, cyclobutyl, ethenyl, 3-propenyl, 1-propenyl, isopropenyl, 3-butenyl, 4-butenyl, 3-propynyl, 3-butynyl, 4-butynyl, diazirinyl, aziridinyl, urazolyl, azetidinyl, pyrazolidinyl, imidazolidinyl, oxazolidinyl, isoxazolinyl, isoxazolyl, thiazolidinyl, isothiazolyl, isothiazolinyl, oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, or piperidin-2-onyl (valerolactam).

The compound wherein R₂ is NH₂⁺, OH, SH, NOH, NHNH₂, NHNH₃⁺, CO₂H, SO₂OH, B(OH)₂, or imidazolium.

In another example, R₃ can be N, NH, NH₂⁺, O, OH, S, SH, C—R₁₃, CH—R₁₃, N—R₁₃, NH—R₁₃, O—R₁₃, or S—R₁₃, wherein R₁₃ is NH₂⁺, NH₃⁺, CO₂H, B(OH)₂, CH(NH₂)₂, C(NH₂)₂⁺, CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present.

In a further example, R₁₀ can be N, NH, O, or S. In a further example, R₇ can be CH.

The cell can be identified as being in need of inhibited gene expression. The cell can be a bacterial cell, for example, and the compound can kill or inhibit the growth of the bacterial cell. The compound and the cell can be brought into contact by administering the compound to a subject. In one example, the compound is not a substrate for enzymes of the subject that have lysine as a substrate. The compound can also not be a substrate for enzymes of the subject that alter lysine. The compound can also not be a substrate for enzymes of the subject that metabolize lysine. The compound can also not be a substrate for enzymes of the subject that catabolize lysine. The cell can be a bacterial cell in the subject, wherein the compound kills or inhibits the growth of the bacterial cell.

Disclosed herein is a compound having the structure of Formula I:

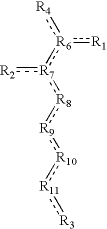

wherein R₂ and R₃ are each independently positively charged, can serve as a hydrogen bond donor, or both,

wherein R₁ is negatively charged, R₄ is negatively charged, or R₁ and R₄ are in a resonance hybrid with a net negative charge,

wherein at least one of R₁ or R₄ can be CH₂, CH₃, NH, O, O⁻, OH, S, S⁻, SH, C—R₁₄, CH—R₁₄, or N—R₁₄, wherein R₁₄ can be CH₂, CH₃, O, O⁻, OH, S, S⁻, or SH,

wherein R₉ can be C, CH, CH₂, NH, O, S, C—R₅, CH—R₅, or N—R₅, wherein R₅ can be methyl, ethyl, propyl, isopropyl, cyclopropyl, butyl, tert-butyl, sec-butyl, iso-butyl, cyclobutyl, ethenyl, 3-propenyl, 1-propenyl, isopropenyl, 3-butenyl, 4-butenyl, 3-propynyl, 3-butynyl, 4-butynyl, diazirinyl, aziridinyl, urazolyl, azetidinyl, pyrazolidinyl, imidazolidinyl, oxazolidinyl, isoxazolinyl, isoxazolyl, thiazolidinyl, isothiazolyl, isothiazolinyl, oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, or piperidin-2-onyl (valerolactam),

wherein R₂ is NH₂⁺, NH₃⁺, OH, SH, NOH, NHNH₂, NHNH₃⁺, CO₂H, SO₂OH, B(OH)₂, or imidazolium,

wherein R₃ can be N, NH, NH₂⁺, NH₃⁺, O, OH, S, SH, C—R₁₃, CH—R₁₃, N—R₁₃, NH—R₁₃, O—R₁₃, or S—R₁₃, wherein R₁₃ is NH₂⁺, NH₃⁺, CO₂H, B(OH)₂, CH(NH₂)₂, C(NH₂)₂⁺, CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present,

wherein R₇ can have an S configuration based on a priority of R₂>R₆>R₈,

wherein R₆, R₇, R₈, R₁₀, and R₁₁ each independently can be C, CH, CH₂, N, NH, O, or S,

wherein [symbol] can each independently represent a single or double bond, and

wherein the compound is not lysine.

R₃ can be positively charged and can serve as a hydrogen bond donor. R₅ can be uncharged. R₉ can be C, O, or S. The pKₐ of R₃ can be 7 or higher. R₁₃ can be positively charged, and can serve as a hydrogen bond donor, or both.

In one example, R₆, R₇, R₈, R₉, R₁₀ and R₁₁ are not all simultaneously C, CH, or CH₂.

In another example, R₁, R₂, R₃, R₄ and R₉ are not simultaneously O, NH₃⁺, NH₃⁺, O and S, respectively. Furthermore, in another example, R₁, R₂, R₃, and R₄ are not simultaneously O, H, NH₃⁺, and O, respectively. In another example, R₁, R₂, R₃, R₄ and R₉ are not simultaneously CO₂⁻, NH₃⁺, NH₃⁺, and H, respectively. In a further example, R₁, R₂, R₃, R₄ and R₁₁ are not simultaneously O, NH₃⁺, NH₃⁺, O and C—CO₂⁻, respectively. In a further example, R₁, R₂, R₃, and R₄ are not simultaneously NHOH, NH₃⁺, NH₃⁺, O and S, respectively.

In one example, R₉ can be NH, O, S, C—R₅, CH—R₅, or N—R₅, wherein R₅ is methyl, ethyl, propyl, isopropyl, cyclopropyl, butyl, tert-butyl, sec-butyl, iso-butyl, cyclobutyl, ethenyl, 3-propenyl, 1-propenyl, isopropenyl, 3-butenyl, 4-butenyl, 3-propynyl, 3-butynyl, 4-butynyl, diazirinyl, aziridinyl, urazolyl, azetidinyl, pyrazolidinyl, imidazolidinyl, oxazolidinyl, isoxazolinyl, isoxazolyl, thiazolidinyl, isothiazolyl, isothiazolinyl, oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, or piperidin-2-onyl (valerolactam).

The compound wherein R₂ is NH₂⁺, OH, SH, NOH, NHNH₂, NHNH₃⁺, CO₂H, SO₂OH, B(OH)₂, or imidazolium.

In another example, R₃ can be N, NH, NH₂⁺, O, OH, S, SH, C—R₁₃, CH—R₁₃, N—R₁₃, NH—R₁₃, O—R₁₃, or S—R₁₃, wherein R₁₃ is NH₂⁺, NH₃⁺, CO₂H, B(OH)₂, CH(NH₂)₂, C(NH₂)₂⁺, CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present.

In a further example, R₁₀ can be N, NH, O, or S. In a further example, R₇ can be CH.

Further disclosed is a composition comprising the compound described above and a regulatable gene expression construct comprising a nucleic acid molecule encoding an RNA comprising a lysine riboswitch operably linked to a coding region, wherein the lysine riboswitch regulates expression of the RNA, wherein the lysine riboswitch and coding region are heterologous. The lysine riboswitch can produce a signal when activated by the compound. For example, the riboswitch can change conformation when activated by the compound, and the change in conformation can produce a signal via a conformation dependent label. Furthermore, the riboswitch can change conformation when activated by the compound, wherein the change in conformation causes a change in expression of the coding region linked to the riboswitch, wherein the change in expression produces a signal. The signal can be produced by a reporter protein expressed from the coding region linked to the riboswitch.

Also disclosed is a method comprising: (a) testing the compound as described above for inhibition of gene expression of a gene encoding an RNA comprising a lysine riboswitch, wherein the inhibition is via the lysine riboswitch, and (b) inhibiting gene expression by bringing into contact a cell and a compound that inhibited gene expression in step (a), wherein the cell comprises a gene encoding an RNA comprising the lysine riboswitch, wherein the compound inhibits expression of the gene by binding to the lysine riboswitch.

Further disclosed is a method of killing bacteria, comprising contacting the bacteria with a compound disclosed above. Disclosed herein is also a method of inhibiting growth of a cell, such as a bacterial cell, that is in a subject, the method comprising administering an effective amount of a compound as disclosed herein to the subject. This can result in the compound being brought into contact with the cell. The subject can have, for example, a bacterial infection, and the bacterial cells can be the cells to be inhibited by the compound. The bacteria can be any bacteria. Bacterial growth can also be inhibited in any context in which bacteria are found. For example, bacterial growth in fluids, biofilms, and on surfaces can be inhibited. The compounds disclosed herein can be administered or used in combination with any other compound or composition. For example, the disclosed compounds can be administered or used in combination with another antimicrobial compound.

### EXAMPLES

### Example 1

### Antibacterial Lysine Analogs that Target Lysine Riboswitches

Lysine riboswitches are bacterial RNA structures that sense the concentration of lysine and regulate the expression of lysine biosynthesis and transport genes. Members of this riboswitch class are found in the 5′-untranslated region (5′-UTR) of messenger RNAs, where they form highly selective receptors for lysine. Lysine binding to the receptor stabilizes an mRNA tertiary structure that, in most cases, causes transcription termination before the adjacent open reading frame can be expressed. A lysine riboswitch can be used for antibacterial therapy by designing compounds that bind the riboswitch and suppress lysine biosynthesis and transport genes. As a test of this strategy, several lysine analogs that bind to riboswitches and inhibit bacterial growth have been identified. These results indicate that riboswitches can serve as antibacterial drug targets.

In one example, a riboswitch-targeting compound is dissimilar to the natural metabolite so that the drug can neither serve as a nutritional supplement for the pathogen, nor interact with host enzymes that process the natural metabolite. In order to identify functional groups where modifications to lysine would be tolerated by lysine riboswitch receptors, 12 lysine analogs were evaluated for their ability to bind the riboswitch receptor from B. subtilis (FIG. 2a). The equilibrium dissociation constant (K_{D}) for each compound was established by conducting in-line probing assays (Soukup 1999) with the 179-nucleotide receptor domain (termed 179 lysC) of the riboswitch (FIG. 2b). In-line probing reveals the ability of each internucleotide linkage to undergo self-cleavage through an S_{N}2P mechanism. As previously reported (Sudarsan 2006) there are three regions of the receptor (A, B, and C, FIG. 2b) where the extent of cleavage is reduced, compared to the pattern in the absence of an added compound, indicating that the RNA undergoes a structural change upon ligand binding. Quantitation of the fraction of RNAs cleaved at each region as a function of ligand concentration gives a reasonable measure of K_{D} (FIG. 2c).

Five of the 12 analogs tested bind to 179 lysC with K_{D} values within 40 fold of the K_{D} for lysine (360 nM), revealing that the riboswitch can tolerate chemical modifications at certain positions of its ligand. The observation that derivatives 1, 2, 4, and AEC (Sudarsan 2006) bind well indicates that additional modifications of various sizes at the C4 position of lysine are likely to be tolerated by the receptor and may even increase the affinity of the interaction. A large functional group can also be accommodated on N6 (compounds 3 and 7), as long as this amine still carries a hydrogen bond donor and a positive charge at neutral pH. When N6 lacks a hydrogen bond donor (12) or has a pKₐ less than 7 (6), no binding is observed. The poor affinity of 8 is consistent with the previous observation that 5-hydroxylysine also fails to bind to the riboswitch (Sudarsan 2006) showing that the RNA cannot tolerate bulky C5 modifications to the ligand. Compounds 9 and 10 have large modifications to the site chain atoms that add bulk and that are expected to produce a pKₐ less than 7 at the amine equivalent to N6. These differences can be sufficient to explain their poor affinities, although other effects such as restricted conformation of the ligand can also hinder binding. Finally, the riboswitch does not tolerate modifications at N2 (5, 11), perhaps due to steric clash or to a change in the ionic character of the nitrogen. Since removing N2 ablates binding (Sudarsan 2006) either the charge or the hydrogen bonding character of N2 is needed for binding. The importance of the carboxylate, the stereochemistry at C2, and the length of the amino acid side chain were previously established (Sudarsan 2006). A summary of the molecular recognition determinants for ligand binding to lysine riboswitch receptors is depicted in FIG. 2d.

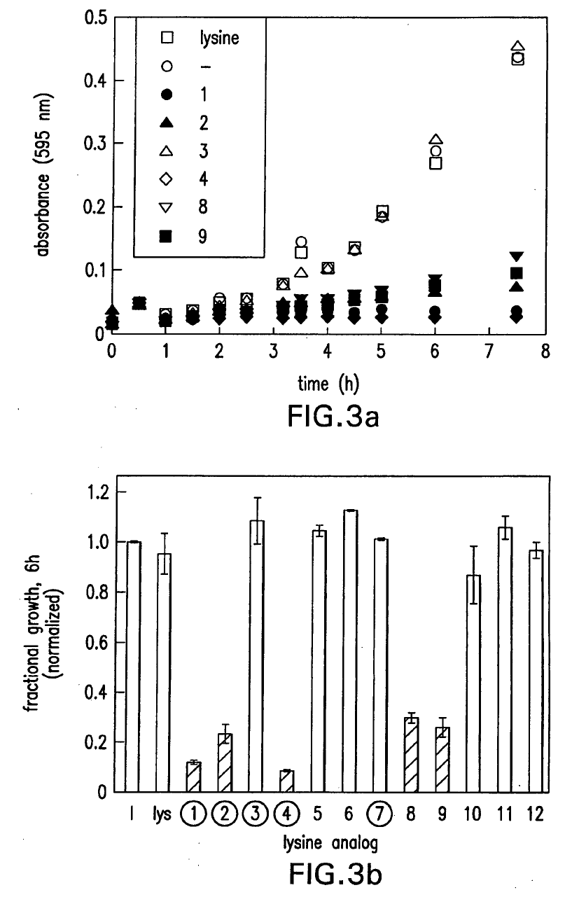

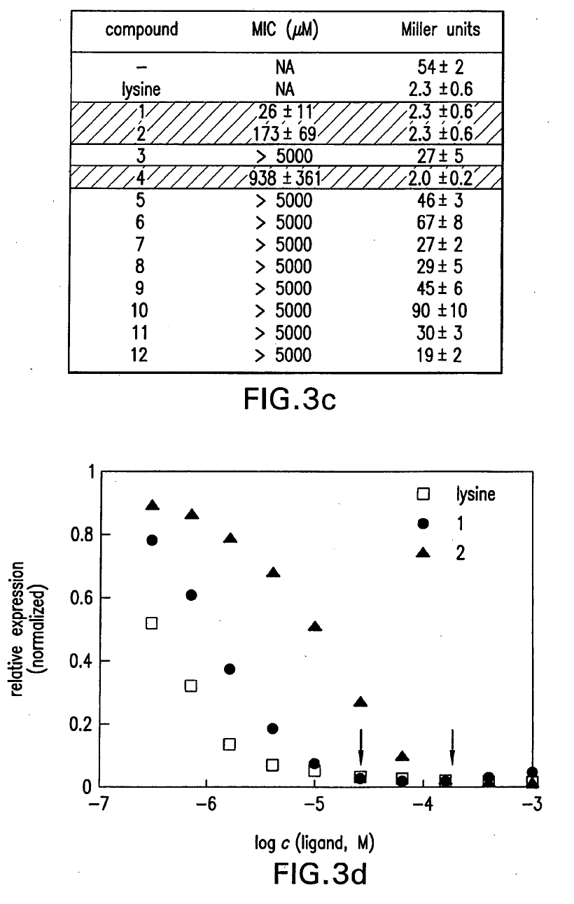

It was next determined whether any of the analogs inhibit the growth of B. subtilis. At 100 μM in a chemically-defined minimal medium (see Methods), five compounds slow bacterial growth (FIG. 3a,b). Of these, only 1, 2, and 4 completely inhibit bacterial growth for 24 h (FIG. 3c). Although 3 binds strongly to the lysine aptamer, this analog is the only compound examined in this study other than lysine that supports the growth of a lysine auxotroph strain (1A40) of B. subtilis (Supplementary FIG. 3). Therefore, it appears that 3 is most likely serving as a fortuitous precursor for lysine production, and this unexpected metabolic conversion circumvents toxicity. Compound 7 also fails to inhibit cell growth despite its strong binding to 179 lysC. 7 appears to be chemically modified by the bacterium, or perhaps it cannot gain entry to the cell. Either explanation is consistent with the observation that 7 does not repress riboswitch-mediated reporter gene expression (FIG. 3c).

Compounds 8 and 9 are rejected by the lysine aptamer when tested in vitro (FIG. 2a), and they do not repress expression of a reporter gene controlled by the lysC riboswitch. However, both compounds exhibit a modest level of growth inhibition activity (FIG. 3a,b). These findings indicate that 8 and 9 inhibit bacterial growth by a mechanism that does not involve the lysine riboswitch. Consistent with this is the observation that 1 and 2 also inhibit the formation of viable spores whereas 8 and 9 do not (Supplementary Table 2), showing that compounds that trigger riboswitch function affect cellular processes that are distinct from those that do not bind the riboswitch.

One likely explanation for the action of 1 and 2 is that they inhibit growth and sporulation by binding to the lysine riboswitch and repressing lysC, thereby depleting the bacteria of lysine and 2,3-dihydropicolinate. An alternate possibility is that they are incorporated into proteins where they disrupt functional interactions. Arguments have been presented for both possibilities as explanations for why L-2-aminoethyl-cysteine (AEC) inhibits bacterial growth (Sudarsan 2006; Grundy 2003). Therefore, the mechanism of growth inhibition for 1, 2, and 4 were more carefully evaluated.

To determine if the lysine analogs repress gene expression, a bacterial strain was constructed in which a copy of the lysC lysine riboswitch was cloned upstream of a lacZ reporter gene and transformed into the amyE locus of B. subtilis. As expected, β-galactosidase expression is strongly repressed by lysine (FIG. 3c). Among the lysine analogs, only 1, 2, and 4 significantly repress β-galactosidase expression, confirming that they can repress natural lysC and, most likely, yvsH expression.

The minimal inhibitory concentration (MIC) of 1, 2, and 4 that prevent growth of B. subtilis are similar to the MIC measured for AEC on this and other bacteria (reference Japanese paper). Notably, the relative concentrations of lysine, 1, and 2 required to completely repress expression (FIG. 3d) correspond well with their relative K_{D} and MIC values. Furthermore, at their respective MICs, 1 and 2 completely repress reporter gene expression. The strong correlation among K_{D}, reporter gene repression, and antibacterial activity is consistent with a mechanism wherein riboswitch-mediated gene repression is responsible for the inhibitory activity of these compounds.

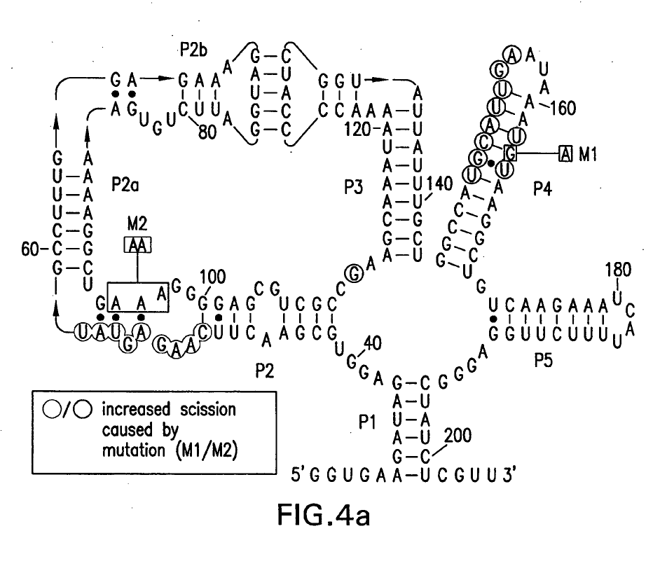

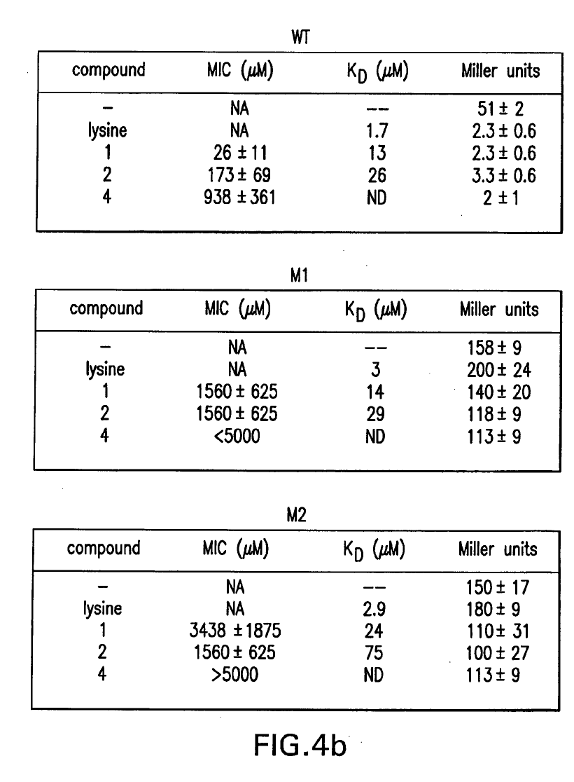

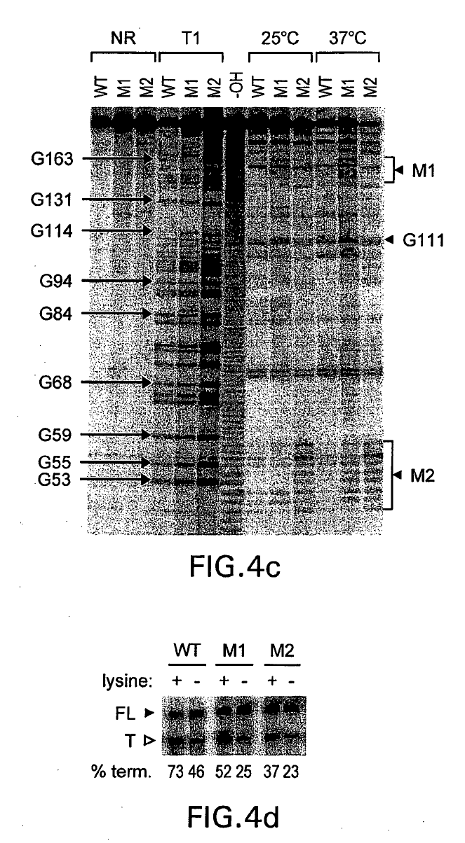

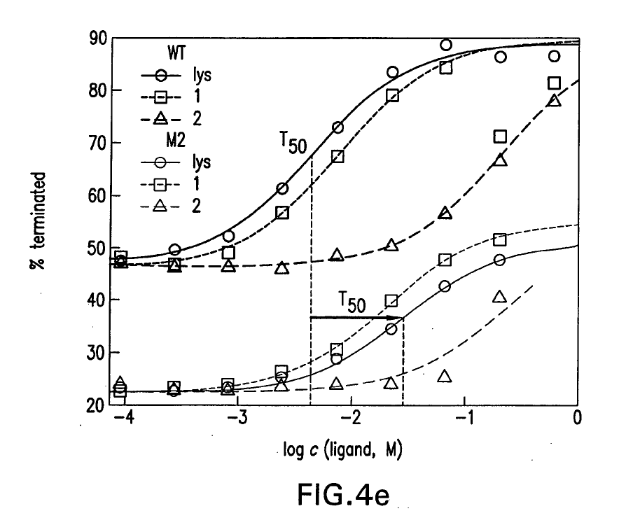

To more fully characterize the mechanism of inhibition, B. subtilis strains were cultivated that are resistant to 2. Resistance to 2 was examined because this compound is a commercially available representative of the compounds that exhibit strong binding to the riboswitch and a good MIC value. Using serial passage (see Methods), 24 bacterial colonies were isolated that exhibit at least 9-fold higher MIC values for 2, and DNAs corresponding to the lysC and yvsH riboswitches from these resistant bacteria were amplified and sequenced. Not surprisingly, no mutations were observed in the yvsH riboswitches of these bacteria. Since these resistant bacteria were evolved in a defined minimal media without lysine supplementation, there was no selective pressure to derepress the expression of the lysine transporter coded by yvsH. Remarkably, every resistant colony had a single mutation in the lysC riboswitch (FIG. 4a). Among the resistant colonies, 21 had a G to A mutation in P4 (M1), and three had an A deletion in the loop E motif of P2 (M2). Importantly, both mutations also confer resistance to 1 and 4 (FIG. 4b), implying that the compounds might have a common mechanism of action. When cloned upstream of the β-galactosidase reporter, constructs containing either the M1 or M2 mutation derepress gene expression, even at lysine concentrations as high as 5 mM. Moreover, because fully active β-galactosidase is still expressed when cells are grown in the presence of high concentrations of 1 and 2, it is unlikely that incorporation of these compounds into proteins is the cause for growth inhibition.

To understand how the mutations disable the lysine riboswitch, in-line probing assays were performed with the lysC receptor region carrying the M1 or M2 mutations. In the absence of ligand, the M1 mutation destabilizes P4 at room temperature and this effect is more pronounced at 37° C. (FIG. 4c). Likewise, in the absence of ligand, the M2 mutation destabilizes the loop E structure. Surprisingly, these structural defects only modestly decrease the K_{D} for lysine (FIG. 4b). It is possible that, like other riboswitches, (Wickisier 2005₁; Wickisier 2005₂) the speed at which the ligand binds the lysine riboswitch, rather than its binding affinity, determines whether gene repression occurs. If that is the case, the mutations disrupt gene regulation by slowing ligand association.

To directly investigate how the M1 and M2 deformations alter gene regulation, in vitro transcription assays were conducted with each variant. In the absence of a ligand, both mutations decrease the termination efficiency compared to the wild-type riboswitch (FIG. 4d). Most likely, each mutation causes a fraction of the receptors to fold into an inactive form that can not form the terminator structure. This effect is not rescued by adding a saturating concentration of lysine (FIG. 4d,e) supporting the assertion that a fraction of the mutated riboswitches is inactive. In addition to causing a folding defect, the mutations result in an increased concentration of ligand needed to induce termination (T₅₀, FIG. 4e). This shows that each mutation also affects ligand binding by the receptor, even within the properly folded population. Because the in vitro binding affinities are not dramatically shifted, this effect most likely reflects a decreased rate of ligand association. In summary, the results indicate that mutations that impair the gene regulatory function of a lysine riboswitch confer resistance to antibacterial lysine analogs.

Collectively, these data demonstrate that the antibacterial lysine mimetics 1, 2, and 4 function, at least in part, by lysine riboswitch-mediated repression of aspartokinase II in B. subtilis.

These compounds can also inhibit bacterial growth in an infection setting. Because many bacteria have an isozyme for aspartokinase II that is not regulated by the riboswitch (FIG. 6), (Zhang 1990) full repression of lysC might not sufficiently quell lysine biosynthesis to inhibit growth within a host environment. However, in a few species, such as Bacillus cereus and Bacillus anthracis, an additional copy of the riboswitch regulates the diaminopimelate decarboxylase gene (lysA in B. subtilis, FIG. 1b), for which no alternate pathway exists (Rodionov 2003). Accordingly, 1 inhibits the growth of B. cereus 13-fold more severely than that of B. subtilis in minimal medium, implying that repression of genes that are more critical to survival is more detrimental to growth. Regardless, neither B. subtilis nor B. anthracis is inhibited by 512 μg ml⁻¹ of 1, or 2 in rich media (see Methods for details). Most likely, dipeptide (Higgins 1986) or other non-regulated amino acid transporters can supply enough lysine from the media, even when lysine biosynthesis is completely repressed. This result shows that compounds that exclusively target the lysine riboswitch are not necessarily potent against pathogens that can glean lysine from the host.

In conclusion, evidence is provided that the lysine riboswitch can serve as an antibacterial drug target in minimal media. It was also found that AEC, originally characterized in 1958 (Shiota 1958) inhibits bacterial growth by targeting the lysine riboswitch (Sudarsan 2006). Combined with the recent discovery that the antibacterial activity of pyrithiamin, also established decades ago (Woolley 1943) targets the thiamine pyrophosphate-binding riboswitch (Sudarsan 2005) this work underscores the generality of targeting riboswitches with antibacterial drugs.

### Methods

Chemicals, oligonucleotides, and bacterial strains. L-lysine, L-4-oxalysine, L-homoarginine, L-N²-acetyllysine, L-N⁶-acetyllysine, L-N⁶-1-iminoethyllysine, L-3-aminotyrosine, L-2-amino-3-(2-aminobenzoyl)-propionic acid, and L-N⁶-trimethyllysine were purchased from Sigma. DL-trans-2,6-diamino-4-hexenoic acid, DL-5-oxolysine, and L-N²-methyllysine were purchased from Bachem. Decoyinine was purchased from MP Biomedicals. Oligonucleotides were synthesized by the HHMI Keck Foundation Biotechnology Resource Center at Yale University. All B. subtilis strains were obtained from the Bacillus Genetic Stock Center (The Ohio State University), with the exception of the M1 and M2 strains that were generated in this study.

L-3-[(2-aminoethyl)-sulfonyl]-alanine was prepared similarly to a previously described method (Toennies 1941). Perchloric acid (70%, 4.1 ml, 47.5 mmol) was added to a solution of ammonium molybdate (586 mg, 3.0 mmol) in 15 ml of water, and the solution was heated at 100-110° C. until a white solid formed. After cooling to 25° C., the mixture was filtered and the filtrate was treated with L-2-aminoethylcysteine (300 mg, 1.5 mmol) followed by hydrogen peroxide (30%, 11.7 ml, 0.122 mol). The mixture was stirred at 25° C. overnight and loaded into Dowex 50WX resin (H⁺ form). After washing the resin with H₂O, the product was collected by eluting with 2N NH₄OH and then concentrated in vacuo to give the product (304.4 mg, 87.5%): ¹H NMR (400 MHz, D₂O) δ 2.81 (m, 2H), 3.04 (m, 2H), 3.16 (t, 2H), 3.88 (t, 1H).

In-line probing assays. The 179 lysC RNA construct used for in-line probing assays was prepared by in vitro transcription using a template generated from whole-cell PCR of the appropriate B. subtilis strains (1A1 or resistant strains). RNA transcripts were dephosphorylated, 5′-³²P-labeled, and subsequently subjected to in-line probing using protocols similar to those described previously (Soukup 1999). For each reaction, approximately 1 nM of labeled RNA was incubated for 39-48 h at room temperature or 16-20 h at 37° C. in a 10 μl solution containing 50 mM Tris (pH 8.3 at 25° C.), 20 mM MgCl₂, and 100 mM KCl in the absence or presence of 1 nM to 6 mM of lysine or each analog as indicated for each experiment. Denaturing 10% polyacrylamide gel electrophoresis (PAGE) was used to separate spontaneous cleavage products, which were detected and quantitated using a GE Healthcare PhosphorImager and ImageQuant NT software.

The K_{D} for each ligand was derived by quantifying the amount of RNA cleaved at each nucleotide position over a range of ligand concentrations. For each region where modification was observed (A, B, and C in FIG. 2b), the fraction of RNA cleaved at each ligand concentration was calculated by assuming that the maximal extent of cleavage is observed in the absence of ligand and the minimal cleavage is observed in the presence of the highest ligand concentration. The apparent K_{D} was determined by fitting the plot of the fraction cleaved, x, versus the ligand concentration, [L], to the following equation: χ=K_{D}/([L]+K_{D}), using SigmaPlot 9 software.

Antibacterial activity assays. Bacterial growth curves were determined by diluting an overnight liquid culture of B. subtilis 168 (1A1) to a concentration at A₅₉₅ of 0.1 in 150 μl of a chemically defined minimal media (Anagnostopoulos 1961) “GMM” (0.5% w/v glucose, 2 g/l (NH₄)₂SO₄, 14 g/l K₂HPO₄, 6 g/l KH₂PO₄, 1 g/l sodium citrate, 0.2 g/l MgSO₄.7H₂O, 5 μM MnCl₂, 0.5 mM CaCl₂, 2.5 mM MgCl₂), supplemented with 50 μg/ml tryptophan and the indicated compound, and quantitating the A₅₉₅ at given time points. MIC values were determined as recommended by CLSI guidelines (Clinical and Laboratory Standards Institute, Methods for dilution antimicrobial susceptibility tests for bacteria that grow aerobically—Ninth ed: Approved Standard: M7-A7, CLSI Wayne, Pa. 2006). Assays to establish the efficacy of compounds against B. cereus and B. anthracia growth in were conducted by Micromyx (Kalamazoo, Mich.) using Mueller-Hinton II media as recommended by CLSI guidelines.

Sporulation assays. Sporulation effects were determined as described elsewhere (Lazazzera 1997). Briefly, an overnight culture of B. subtilis 168 strain 1A1 in GMM was diluted by a factor of 30 into GMM supplemented with 20 mM glutamate and grown at 37° C. with shaking to an A₆₀₀ of 0.5-0.7. After adding an aliquot of the indicated lysine analog, sporulation was induced by adding decoyinine to a final concentration of 500 μg/ml. After growing for an additional 22-24 h, the efficiency at which cultures formed viable spores was measured by plating an aliquot of the culture onto TBAB either before or after heating at 80° C. for 20 min and quantifying the surviving colonies.

In vivo reporter gene expression assays. The lysC 5′-UTR from a wild-type B. subtilis strain or a representative of the resistant M1 or M2 strains was PCR amplified, ligated upstream of a β-galactosidase reporter gene, and integrated into the genome of B. subtilis following methods described previously (Sudarsan 2006). Briefly, nucleotides—399 to ˜17 relative to the lysC translation start site were amplified as an EcoRI-BamHI fragment by whole-cell PCR of each strain. The PCR products were cloned into pDG1661 (Guerot-Fleury 1996) immediately upstream of the lacZ reporter gene, with integrity confirmed by sequencing. The resulting pDG1661 variants were transformed into the amyE locus of B. subtilis strain 1A40 using standard protocols (Jarmer 2002) and correct transformants were selected by screening for chloramphenicol (5 μg/ml) resistance and spectinomycin (50 μg/ml) sensitivity.

β-galactosidase expression levels of each strain were measured as described previously (Sudarsan 2006). Briefly, cells were grown overnight with shaking at 37° C. in GMM supplemented with 50 μg/ml each of tryptophan, methionine and lysine. The following day, the cells were centrifuged, and the pellet was resuspended in GMM supplemented with 50 μg/ml each of tryptophan and methionine. The resuspended cells were diluted by a factor of 10 into the same medium supplemented with lysine or a lysine analog at the indicated concentration. After growing for 3 h at 37° C., β-galactosidase assays were performed using a standard protocol.

β-galactosidase expression as a function of ligand concentration was determined similarly, except that cell growth and quantitation of expression were performed in 96-well microplates. Briefly, a culture of each strain at a starting A₅₉₅ of 0.6 was grown for 3 h at 37° C. in 150 μl of GMM supplemented with 50 μg/ml tryptophan and methionine and varying ligand concentrations. After recording the absorbance at 595 nm using a Beckman Coulter DTX 880 plate reader, the cells were permeabilized by mixing 100 of each culture into 0.5 ml Z-buffer (100 mM Na₂HPO₄ [pH 7.0 at 25° C.], 10 mM KCl, 1 mM MgSO₄, and 50 mM β-mercaptoethanol), 10 μl 0.1% SDS, and 40 μl of chloroform per well of a polypropylene microplate. After allowing the solution to settle, 150 μl was transferred to a separate plate and incubated for 20 min at 25° C. with 25 μl ortho-nitrophenyl-β-galactoside, followed by quantitation of the absorbance at 414 nm and 550 nm and calculation of Miller units as previously reported for riboswitch gene repression assays (Sudarsan 2006).

Evolution of L-4-oxalysine-resistant strains. Resistant mutants were cultivated using serial passage (Kawasaki 1969). A fresh overnight culture of B. subtilis 168 strain 1A1 in GMM supplemented with 50 μg/ml tryptophan was diluted by a factor of 100 into the same medium with 100 μM L-4-oxalysine (2) and grown at 37° C. with shaking, until the culture reached saturation. A 10 μl aliquot was then diluted into 1 ml GMM containing 100 μM L-4-oxalysine, and this process was repeated until, after six passages, the culture reached saturation at the same rate as a culture with no added compound. After plating onto tryptone blood agar base (TBAB), the genomic regions encompassing the 5′-UTRs of the lysC gene (−486 to +110 relative to the translation start site) and the yvsH gene (−376 to +1564) were amplified and sequenced from 24 of the resistant isolates.

In vitro transcription termination assays. Single-round transcription termination assays were conducted following protocols adapted from a previously described method (Landick 1996). The DNA templates covered the region −390 to −17 (relative to the start of translation) of the B. subtilis lysC gene, with a point mutation (C6G of the RNA) to eliminate C residues on the nascent RNA before nucleotide position 17 and were generated by whole-cell PCR of B. subtilis 168 strain 1A1 or the corresponding resistant strains. To initiate transcription and form C17-halted complexes, each sample was incubated at 37° C. for 10 min and contained 1 pmole DNA template, 0.2 mM ApA dinucleotide, 1 μM each of ATP, GTP, and UTP, plus 2 μCi 5′-[α-³²P]-UTP, and 0.4 U E. coli RNA polymerase holoenzyme (Epicenter) in 10 μl of 80 mM Tris-HCl (pH 8.0 at 26° C.), 20 mM NaCl, 14 mM MgCl₂, 0.1 mM EDTA and 0.01 mg/ml BSA. Halted complexes were restarted by the simultaneous addition of 10 μM each of the four NTPs, 0.2 mg/ml heparin to prevent re-initiation, and different concentrations of ligand as indicated to yield a final volume of 12.5 μl in a buffer containing 150 mM Tris-HCl (pH 8.0 at 26° C.), 20 mM NaCl, 14 mM MgCl₂, 0.1 mM EDTA and 0.01 mg/ml BSA. Reactions were incubated for an additional 20 min at 37° C., and the products were separated by denaturing 10% PAGE followed by quantitation as described above.

TABLE 1
The distribution of lysine riboswitches among bacterial species. Based
on Rodionov et al (Rodionov 2003). Gene names are as shown in FIG. 6.

| Species in which | | | |
|---|---|---|---|
| lysine riboswitches | | | |
| have been | Number of | Number of genes | Genes regulated |
| positively | lysine | regulated by lysine | by lysine |
| identified | riboswitches | riboswitches | riboswitches |
| Actinobacillus | 1 | 1 | lysW |
| pleuropneumoniae | | | |
| Bacillus anthracis | 4 | 4 | lysA, yvsH, lysP, lysC |
| Bacillus cereus | 4 | 4 | lysC, yvsH, lysA, lysP |
| Bacillus clausii | 2 | 2 | dapA, lysW |
| Bacillus halodurans | 3 | 3 | dapA, lysW, lysC, |
| Bacillus | 2 | 2 | lysC, yvsH |
| licheniformis | | | |
| Bacillus subtilis | 2 | 2 | lysC, yvsH |
| Bacillus | 4 | 4 | lysA, dapG, lysP, yvsH |
| thuringiensis | | | |
| Clostridium | 3 | 3 | dapA, lysA, lysP |
| acetobutylicum | | | |
| Clostridium | 3 | 3 | lysA, lysP, lysW |
| perfringens | | | |
| Clostridium tetani | 2 | 2 | lysA, lysW |
| Clostridium | 1 | 1 | dapA |
| thermocellum | | | |
| Desulfitobacterium | 1 | 1 | lysA |
| hafniense | | | |
| Enterococcus | 1 | 1 | lysXY |
| faecalis | | | |
| Erwinia carotovora | 1 | 1 | lysC |
| Escherichia coli | 1 | 1 | lysC |
| Exiguobacterium sp. | 3 | 3 | lysC, lysW, lysP |
| Fusobacterium | 1 | 1 | hypothetical protein, unknown |
| nucleatum | | | function |
| Geobacillus | 1 | 1 | lysP |
| kaustophilus | | | |
| Haemophilus | 2 | 2 | lysW, lysC |
| ducreyi | | | |
| Haemophilus | 1 | 1 | lysW |
| influenzae | | | |
| Haemophilus | 1 | 1 | lysW |
| somnus | | | |
| Idiomarina | 1 | 1 | lysC |
| loihiensis | | | |
| Lactobacillus | 2 | 2 | lysA, putative glutamine ABC |
| acidophilus | | | transporter |
| Lactobacillus casei | 3 | 3 | lysA, lysXY, lysP |
| Lactobacillus | 1 | 1 | lysT (COG 0765) |
| delbrueckii | | | |
| Lactobacillus | 1 | 1 | lysXY |
| gasseri | | | |
| Lactobacillus | 1 | 1 | hypothetical transport protein |
| johnsonii | | | |
| Lactobacillus | 1 | 1 | lysP |
| plantarum | | | |
| Lactococcus lactis | 2 | 2 | lysP, dapA |
| Leuconostoc | 3 | 8 | dapF*-asd, lysA-dapD-ykuR- |
| mesenteroides | | | dapA-dapB, lysP |
| Listeria innocua | 1 | 1 | lysP |
| Listeria | 1 | 1 | lysP |
| monocytogenes | | | |
| Moorella | 1 | 1 | dapB |
| thermoacetica | | | |
| Oceanobacillus | 2 | 2 | lysA, lysW |
| iheyensis | | | |
| Oenococcus oeni | 2 | 8 | lysA-dapD-ykuR-dapA-dapB- |
| | | | asd-patA lysXY |
| Pasteurella | 1 | 1 | lysW |
| multocida | | | |
| Pediococcus | 2 | 2 | lysA, lysP |
| pentosaceus | | | |
| Shewanella | 2 | 2 | lysC, lysW |
| oneidensis | | | |
| Shigella flexneri | 1 | 1 | lysC, |
| Solibacter usitatus | 1 | 1 | dapA |
| Staphylococcus | 2 | 2 | lysC, lysP |
| aureus | | | |
| Staphylococcus | 2 | 2 | lysC, lysP |
| epidermidis | | | |
| Thermoanaerobacter | 2 | 2 | lysA, yvsH |
| tengcongensis | | | |
| Thermotoga | 1 | 6 | asd-dapF-dapF-dapB-dapD- |
| maritima | | | lysC |
| Vibrio cholerae | 3 | 3 | lysC, lysW, lysW2 |
| Vibrio fischeri | 2 | 2 | lysC, lysW |
| Vibrio | 2 | 2 | lysC, lysW |
| parahaemolyticus | | | |
| Vibrio vulnificus | 2 | 2 | lysC, lysW |

*Putative assignment of gene function based on sequence comparison.²

TABLE 2
Sporulation efficiency of B. subtilis when grown in the presence
of lysine analogs, determined as described elsewhere. (Lazazzera
1997). The data are expressed as the number of viable colony
forming spores formed after heat shock relative to the number of
viable colony forming units present before heat shock. Four analogs
that affected cell growth were tested for sporulation effects to provide
two examples of compounds that are bound by the riboswitch and two
examples of compounds that are not bound by the riboswitch.

% Viable
spore formation

| Analog | Concentration (mM) | WT | M2 |
|---|---|---|---|
| No ligand | — | 71 | 19 |
| 1 | 0.2 | 7 | ND¹ |
| | 2 | 0 | 17 |
| 2 | 0.2 | 71 | ND |
| | 2 | 6 | 65 |
| 8 | 2 | 47 | 9 |
| 9 | 2 | 75 | 75 |

¹ND designates not determined.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a riboswitch” includes a plurality of such riboswitches, reference to “the riboswitch” is a reference to one or more riboswitches and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps.

Those skilled in the art will recognize, or be able to ascertain using no more than routine experimentation, many equivalents to the specific embodiments of the method and compositions described herein. Such equivalents are intended to be encompassed by the following claims.

### REFERENCES

1. Wolfson, W. Holding back the tide of antibiotic resistance. Chem. Biol. 13, 1-3 (2006).
2. D'Costa, V. M., McGrann, K. M., Hughes, D. W. & Wright, G. D. Sampling the antibiotic resistome. Science 311, 374-377 (2006).
3. Winkler, W. C. & Breaker, R. R. Regulation of bacterial gene expression by riboswitches. Ann. Rev. Microbiol. 59, 487-517 (2005).
4. Mandal, M. & Breaker, R. R. Gene regulation by riboswitches. Nature Reviews Molecular Cell Biology 5, 451-463 (2004).
5. Sudarsan, N., Wickiser, J. K., Nakamura, S., Ebert, M. S. & Breaker, R. R. An mRNA structure in bacteria that controls gene expression by binding lysine. Genes. Dev. 17, 2688-2697 (2003).
6. Grundy, F., Lehman, S. & Henkin, T. The L box regulon: Lysine sensing by leader RNAs of bacterial lysine biosynthesis genes. Nucleic Acids Res. 100, 12057-12062 (2003).
7. Rodionov, D. A., Vitreschak, A. G., Mironov, A. A. & Gelfand, M. S. Regulation of lysine biosynthesis and transport genes in bacteria: yet another RNA riboswitch? Nucleic Acids Res. 31, 6748-6757 (2003).
8. Wimberly, B., Varani, G. & Tinoco, I. J. The conformation of loop E of eukaryotic 5S ribosomal RNA. Biochemistry 32, 1078-1087 (1993).
9. Klein, D. J., Schmeing, T. M., Moore, P. B. & Steitz, T. A. The kink-turn: a new RNA secondary structure motif. EMBO J. 20, 4214-4221 (2001).
10. Hutton, C. A., Southwood, T. J. & Turner, J. J. Inhibitors of lysine biosynthesis as antibacterial agents. Mini. Rev. Med. Chem. 3, 115-127 (2003).
11. Bugg, T. D. H. & Brandish, P. E. From peptidoglycan to glycoproteins: common features of lipid-linked oligosaccharide biosynthesis. FEMS Microbiol. Lett. 119, 255-262 (1994).
12. Sudarsan, N., Cohen-Chalamish, S., Nakamura, S., Emilsson Gail, M. & Breaker, R. R. Thiamine pyrophosphate riboswitches are targets for the antimicrobial compound pyrithiamine. Chem. Biol. 12, 1253-1358 (2005).
13. Woolley, D. W. & White, A. G. C. Selective reversible inhibition of microbial growth with pyrithiamine. J. Exp. Med. 78, 489-497 (1943).
14. Shiota, T., Folk, J. E. & Tietze, F. Inhibition of lysine utilization in bacteria by S-(beta-aminoethyl) cysteine and its reversal by lysine peptides. Arch. Biochem. Biophys. 77, 372-7 (1958).
15. Lu, Y., Chen, N. Y. & Paulus, H. Identification of aecA mutations in Bacillus subtilis as nucleotide substitutions in the untranslated leader region of the aspartokinase II operon. J. Gen. Microbiol. 92, 23-27 (1991).
16. Patte, J. C., Akrim, M. & Mejean, V. The leader sequence of the Escherichia coli lysC gene is involved in the regulation of LysC synthesis. FEMS Microbiol. Lett. 169, 165-170 (1998).
17. Soukup, G. A. & Breaker, R. R. Relationship between internucleotide linkage geometry and the stability of RNA. RNA 5, 1308-1325 (1999).
18. Wickiser, J. K., Winkler Wade, C., Breaker Ronald, R. & Crothers Donald, M. The Speed of RNA Transcription and Metabolite Binding Kinetics Operate an FMN Riboswitch. Molecular cell 18, 49-60 (2005).
19. Wickiser, J. K., Cheah, M. T., Breaker, R. R. & Crothers, D. M. The Kinetics of Ligand Binding by an Adenine-Sensing Riboswitch. Biochemistry 44, 13404-13414 (2005).
20. Zhang, J. J., Hu, F. M., Chen, N. Y. & Paulus, H. Comparison of the three aspartokinase isozymes in Bacillus subtilis Marburg and 168. J. Bacteriol. 172, 701-708 (1990).
21. Higgins, C. F. & Gibson, M. M. Peptide transport in bacteria. Methods Enzymol. 125, 365-77 (1986).
22. Kobayashi, K. et al. Essential Bacillus subtilis genes. Proc. Natl. Acad. Sci. U.S.A. 100, 4678-4683 (2003).
23. Toennies, G. & Kolb, J. J. Methionine studies. VI. dl-methionine sulfone. J. Biol. Chem. 140, 131-134 (1941).
24. Anagnostopoulos, C. & Spizizen, J. Requirements for transformation in Bacillus subtilis. J. Bacteriol. 81, 741-746 (1961).
25. Clinical and Laboratory Standards Institute. Methods for dilution antimicrobial susceptibility tests for bacteria that grow aerobically—Ninth Edition: Approved Standard: M7-A7. (CLSI, Wayne, Pa., 2006).
26. Landick, R., Wang, D. & Chan, C. L. Quantitative analysis of transcriptional pausing by Escherichi coli RNA polymerase: His leader pause site as a paradigm. Methods Enzyma 274, 334-353 (1996).
27. Guerout-Fleury, A., Frandsen, N. & Stragier, P. Plasmids for ectopic integration into Bacillus subtilis. Gene 180, 57-61 (1996).
28. Jarmer, H., Berka, R., Knudsen, S. & Saxild, H. H. Transcriptome analysis documents induced competence of Bacillus subtilis during nitrogen limiting conditions. FEMS Microbia Lett. 206, 197-200 (2002).
29. Kawasaki, T. & Nose, Y. Thiamine regulatory mutants in Escherichia coli. J. Biochem. (Tokyo) 65, 417-25 (1969).
30. Lazazzera, B. A., Solomon, J. M. & Grossman, A. D. An exported peptide functions intracellularly to contribute to cell density signalling in B. subtilis. Cell 89, 917-925 (1997).
31. Hutton, C. A., Southwood, T. J. & Turner, J. J. Inhibitors of lysine biosynthesis as antibacterial agents. Mini. Rev. Med. Chem. 3, 115-127 (2003).
32. Rodionov, D. A., Vitreschak, A. G., Mironov, A. A. & Gelfand, M. S. Regulation of lysine biosynthesis and transport genes in bacteria: yet another RNA riboswitch? Nucleic Acids Res. 31, 6748-6757 (2003).
33. Steffes, C., Ellix, J., Wu, J. & Rosen, B. P. The lysP gene encodes the lysine-specific permease. J. Bacteria 174, 3242-3249 (1992).
34. Lazazzera, B. A., Solomon, J. M. & Grossman, A. D. An exported peptide functions intracellularly to contribute to cell density signalling in B. subtilis. Cell 89, 917-925 (1997).

We claim:

1. A method of inhibiting gene expression, the method comprising
(a) bringing into contact a compound and a cell,
(b) wherein the compound has the structure of Formula I: 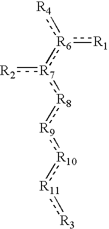
wherein R₂ and R₃ are each independently positively charged, can serve as a hydrogen bond donor, or both,
wherein R₁ is negatively charged, R₄ is negatively charged, or R₁ and R₄ are in a resonance hybrid with a net negative charge,
wherein at least one of R₁ or R₄ is CH₂, CH₃, NH, O, O⁻, OH, S, S⁻, SH, C—R₁₄, CH—R₁₄, or N—R₁₄, wherein R₁₄ is CH₂, CH₃, O, O⁻, OH, S, S⁻, or SH,
wherein R₉ is C, CH, CH₂, NH, O, S, C—R₅, CH—R₅, or N—R₅, wherein R₅ is methyl, ethyl, propyl, isopropyl, cyclopropyl, butyl, tert-butyl, sec-butyl, iso-butyl, cyclobutyl, ethenyl, 3-propenyl, 1-propenyl, isopropenyl, 3-butenyl, 4-butenyl, 3-propynyl, 3-butynyl, 4-butynyl, diazirinyl, aziridinyl, urazolyl, azetidinyl, pyrazolidinyl, imidazolidinyl, oxazolidinyl, isoxazolinyl, isoxazolyl, thiazolidinyl, isothiazolyl, isothiazolinyl, oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, or piperidin-2-onyl (valerolactam),
wherein R₂ is NH₂⁺, NH₃⁺, OH, SH, NOH, NHNH₂, NHNH₃⁺, CO₂H, SO₂OH, B(OH)₂, or imidazolium,
wherein R₃ is N, NH, NH₂⁺, NH₃⁺, O, OH, S, SH, C—R₁₃, CH—R₁₃, N—R₁₃, NH—R₁₃, O—R₁₃, or S—R₁₃, wherein R₁₃ is NH₂⁺, NH₃⁺, CO₂H, B(OH)₂, CH(NH₂)₂, C(NH₂)₂⁺, CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present,
wherein R₇ has an S configuration based on a priority of R₂>R₆>R₈,
wherein R₆, R₇, R₈, R₁₀, and R₁₁ are each independently C, CH, CH₂, N, NH, O, or S,
wherein [symbol] each independently represent a single or double bond,
wherein the compound is not lysine, and
wherein the cell comprises a gene encoding an RNA comprising a lysine-responsive riboswitch, wherein the compound inhibits expression of the gene by binding to the lysine-responsive riboswitch.

2-17. (canceled)

18. The method of claim 1, wherein the cell has been identified as being in need of inhibited gene expression.

19. The method of claim 1, wherein the cell is a bacterial cell.

20. The method of claim 1, wherein the compound kills or inhibits the growth of the cell.

21. The method of claim 1, wherein the compound and the cell are brought into contact by administering the compound to a subject.

22. The method of claim 21, wherein the compound is not a substrate for enzymes of the subject that have lysine as a substrate.

23-25. (canceled)

26. The method of claim 21, wherein the cell is a bacterial cell in the subject, wherein the compound kills or inhibits the growth of the bacterial cell.

27. The method of claim 21, wherein the subject has a bacterial infection.

28. The method of claim 21, wherein the cell contains a lysine riboswitch.

29. The method of claim 21, wherein the compound is administered in combination with another antimicrobial compound.

30. The method of claim 1, wherein the compound inhibits bacterial growth in a biofilm.

31. A compound having the structure of Formula I: 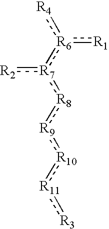
wherein R₂ and R₃ are each independently positively charged, can serve as a hydrogen bond donor, or both,
wherein R₁ is negatively charged, R₄ is negatively charged, or R₁ and R₄ are in a resonance hybrid with a net negative charge,
wherein at least one of R₁ or R₄ is CH₂, CH₃, NH, O, O⁻, OH, S, S⁻, SH, C—R₁₄, CH—R₁₄, or N—R₁₄, wherein R₁₄ is CH₂, CH₃, O, O⁻, OH, S, S⁻, or SH,
wherein R₉ is C, CH, CH₂, NH, O, S, C—R₅, CH—R₅, or N—R₅, wherein R₅ is methyl, ethyl, propyl, isopropyl, cyclopropyl, butyl, tert-butyl, sec-butyl, iso-butyl, cyclobutyl, ethenyl, 3-propenyl, 1-propenyl, isopropenyl, 3-butenyl, 4-butenyl, 3-propynyl, 3-butynyl, 4-butynyl, diazirinyl, aziridinyl, urazolyl, azetidinyl, pyrazolidinyl, imidazolidinyl, oxazolidinyl, isoxazolinyl, isoxazolyl, thiazolidinyl, isothiazolyl, isothiazolinyl, oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, or piperidin-2-onyl (valerolactam),
wherein R₂ is NH₂⁺, NH₃⁺, OH, SH, NOH, NHNH₂, NHNH₃⁺, CO₂H, SO₂OH, B(OH)₂, or imidazolium,
wherein R₃ is N, NH, NH₂⁺, NH₃⁺, O, OH, S, SH, C—R₁₃, CH—R₁₃, N—R₁₃, NH—R₁₃, O—R₁₃, or S—R₁₃, wherein R₁₃ is NH₂⁺, NH₃⁺, CO₂H, B(OH)₂, CH(NH₂)₂, C(NH₂)₂⁺, CNH₂NH₃⁺, C(NH₃⁺)₃, hydroxymethyl, 1-hydroxyethyl, 2-hydroxyethyl, 1,2-dihydroxyethyl, 2-hydroxy-1-methylethyl, 1-hydroxypropyl, 2-hydroxypropyl, 3-hydroxypropyl, 1,3-dihydroxypropyl, 2,3-dihydroxypropyl, 1-hydroxybutyl, 2-hydroxybutyl, 3-hydroxybutyl, 4-hydroxybutyl, 1,4 dihydroxybutyl, 2,4-dihydroxybutyl, 1-hydroxy-2-methylpropyl, 2-hydroxy-2-methylpropyl, 3-hydroxy-2-methylpropyl, 1-hydroxymethyl-1-methylethyl, trishydroxymethylmethyl, thiolmethyl, 1-thiolethyl, 2-thiolethyl, 1,2-dithiolethyl, 2-thiol-1-methylethyl, 1-thiolpropyl, 2-thiolpropyl, 3-thiolpropyl, 1,3-dithiolpropyl, 2,3-dithiolpropyl, 1-thiolbutyl, 2-thiolbutyl, 3-thiolbutyl, 4-thiolbutyl, 1,4 dithiolbutyl, 2,4-dithiolbutyl, 1-thiol-2-methylpropyl, 2-thiol-2-methylpropyl, 3-thiol-2-methylpropyl, 1-thiolmethyl-1-methylethyl, tristhiolmethylmethyl, aminomethyl, 1-aminoethyl, 2-aminoethyl, 1,2-diaminoethyl, 2-amino-1-methylethyl, 1-aminopropyl, 2-aminopropyl, 3-aminopropyl, 1,3-diaminopropyl, 2,3-diaminopropyl, 1-aminobutyl, 2-aminobutyl, 3-aminobutyl, 4-aminobutyl, 1,4 diaminobutyl, 2,4-diaminobutyl, 1-amino-2-methylpropyl, 2-amino-2-methylpropyl, 3-amino-2-methylpropyl, 1-aminomethyl-1-methylethyl, trisaminomethylmethyl, or is not present,
wherein R₇ has an S configuration based on a priority of R₂>R₆>R₈,
wherein R₆, R₇, R₈, R₁₀, and R₁₁ are each independently C, CH, CH₂, N, NH, O, or S,
wherein [symbol] each independently represent a single or double bond,
wherein the compound is not lysine.

32-52. (canceled)

53. A method comprising:
(a) testing the compound of claim 31 for inhibition of gene expression of a gene encoding an RNA comprising a lysine riboswitch, wherein the inhibition is via the lysine riboswitch,
(b) inhibiting gene expression by bringing into contact a cell and a compound that inhibited gene expression in step (a),
wherein the cell comprises a gene encoding an RNA comprising the lysine riboswitch,
wherein the compound inhibits expression of the gene by binding to the lysine riboswitch.

54. The method of claim 1, wherein the compound of formula I is selected from Compounds 1, 2, 3, 4 and 7 of FIG. 2a-1 and 2a-II.3.

55. The method of claim 1, wherein the compound of Formula I is Compounds 1, 2 or 4.

56. The compound of claim 31, wherein the compound of formula I is selected from Compounds 1, 2, 3, 4 and 7 of FIG. 2a-1 and 2a-II.3

57. The compound of claim 31, wherein the compound of Formula I is Compounds 1, 2 or 4.
